(19) United States
(12) Patent Application Publication
(10) Pub. No.: US 2003/0022243 A1
(43) Pub. Date: Jan. 30, 2003

(54) Protein aggregation assays and uses thereof

(76) Inventors: Les Kondejewski, St. Lazare (CA); Avijit Chakrabartty, Vaughan (CA); Xiao-Fei Qi, Toronto (CA); Neil Cashman, Toronto (CA)

Correspondence Address: CLARK & ELBING LLP, 101 FEDERAL STREET, BOSTON, MA, 02110, US

(21) Appl. No.: 10/176,809

(22) Filed: Jun. 20, 2002

Related U.S. Application Data
(60) Provisional application No. 60/299,849, filed on Jun. 20, 2001.

(51) Int. Cl. G01N 33/53; G01N 33/567

(52) U.S. Cl. 435/7.1; 435/7.21

(57) ABSTRACT

This invention features methods for identifying agents that modulate protein aggregation or stabilize protein conformation. Exemplary methods include an in vitro aggregation assay, a native state stabilization assay, a cell-based screening assay, and an animal-based screening assay. These methods can be used to identify agents useful for the treatment of conformational diseases resulting from aggregation of a protein.

### CROSS REFERENCES TO RELATED APPLICATIONS

This application claims benefit of U.S. provisional patent application number 60/299,849 filed on Jun. 20, 2001.

### BACKGROUND OF THE INVENTION

This invention is in the field of screening assays for identifying agents including human pharmaceuticals that modulate protein aggregation or stabilize protein conformation. The invention is applicable for treating a variety of medical disorders resulting from abnormal protein conformation including protein misfolding.

The correctly folded state or so-called native conformation of a protein is often necessary for proper biological function and recognition by other molecules. Abnormal protein conformation including misfolding and aggregation leads to significant loss or alteration of biological activity. Abnormal protein conformation including protein misfolding and aggregation has been identified as the causative agent in a number of human diseases including cystic fibrosis, Alzheimer's disease, prion spongiform encephalopathies such as Creutzfeldt-Jacob disease, and amyotrophic lateral sclerosis (ALS).

ALS is a fatal neuromuscular disease presenting as weakness, muscle atrophy, and spasticity (neurological stiffness). ALS is a result of the degeneration of motor neurons in the brain, brainstem, and spinal cord, producing progressive paralysis of the limbs, and the muscles of speech, swallowing, and respiration. Although death occasionally results shortly after the symptomatic disease, the disease generally ends with respiratory failure secondary to profound generalized and diaphragmatic weakness. Eighty percent of individuals with ALS are dead within two to five years of diagnosis.

Approximately 20,000-30,000 individuals are living with ALS in North America at any given time. The cause of the disease is unknown and ALS may only be diagnosed when the patient begins to experience limp weakness, fatigue and spasticity in the legs, which typifies onset.

Approximately ten percent of all ALS cases are familial (FALS) and a subset of these is a result of dominantly inherited mutations in the gene encoding the enzyme Cu/Zn-superoxide dismutase (SOD-1)(Deng et al., Science 261:1047, 1999; Rosen et al., Nature 362:59, 1993; Shaw et al., Ann. Neurol. 43:490, 1998). SOD-1 is an intracellular enzyme responsible for detoxification of free radicals, catalyzing the breakdown of damaging superoxide anions to hydrogen peroxide and oxygen through a redox cycling of copper bound to the active site:

SOD1-Cu²⁺+2O₂⁻+2H⁺→SOD1-Cu¹⁺+O₂+2H⁺+O₂⁻→SOD1-Cu²⁺+O₂+H₂O₂

Human SOD-1 is 32 kDa homodimeric enzyme that exists in a predominantly β-barrel structure (FIG. 1A). Each subunit possesses one Cu and one Zn atom. In each subunit, the Cu atom is coordinated by 4 histidine residues and is required for the redox reaction whereas the Zn atom is coordinated by 3 histidine residues and one aspartic acid residue and is important in stabilizing the conformation of the active site (FIGS. 1A and 1B).

The finding that many FALS-associated SOD-1 mutants possess full specific activity suggests that the disease is not caused by loss of enzymatic activity. Further support for this idea has come from transgenic mice studies. Transgenic mice harboring FALS-associated SOD-1 mutations develop ALS-like symptoms despite having ample amounts of endogenous mouse SOD-1 enzyme. Furthermore, SOD-1 knockout mice do not develop ALS-like symptoms. Thus, it has been hypothesized that mutations in SOD-1 cause FALS by a gain, not a loss, of function (see, for example, Morrison et al, Brain Res. Rev. 29:121, 1999).

Although a number of alterations in the enzymatic activity of SOD-1 mutants have been observed, the most dramatic gain-of-function exhibited by the SOD-1 mutants is a very high propensity to aggregate. Cells transfected with FALS-associated SOD-1 mutants produce cytoplasmic aggregates composed of the SOD-1 mutant protein; transfections of wild type SOD-1, on the other hand, do not cause such cellular alterations (Koide et al., Neurosci. Lett. 257:29, 1998; Johnston et al., Proc. Natl. Acad. Sci. 97:12571, 2000). Similar aggregates have been reported following introduction of mutant SOD-1 into cultured motor neurons (Durham et al., J. Neuropath. Exp. Neurol. 56:523, 1997). A number of transgenic mice, all expressing a particular FALS-associated SOD-1 mutant and co-expressing different amounts of wild type SOD-1, were shown to uniformly exhibit intracellular SOD-1 aggregation in neural tissue as well as ALS-like symptoms (Bruijn et al., Science 281:1851, 1998). ALS-like symptoms were present regardless of whether wild type SOD-1 expression was elevated or eliminated, suggesting that the aggregates themselves possess toxic properties (Bruijn et al., supra).

The formation of SOD-1 aggregates highlights a common finding in neurodegenerative diseases—that mutant proteins misfold and form intracellular aggregates. Indeed, the recognition of protein misfolding and subsequent aggregation as a mechanism of disease has led to the identification of a number of other diseases recognized in the art as “conformational diseases.” Mechanisms by which SOD-1 aggregates cause toxicity have been proposed. For example, a recent study has shown that protein aggregates themselves have inherent toxicity (Bucciantini et al., Nature, 416:507, 2002). Another hypothesis is that the process of SOD-1 aggregation sequesters other protein components important for neuronal viability (Bruijn et al., supra). Yet another hypothesis is that abnormallyfolded/aggregated SOD-1 can tie up the proteasome pathway required for normal protein turnover, thereby increasing the intracellular content of other misfolded or damaged proteins (Johnston et al., supra). Finally, it has been suggested that through repetitive misfolding, both soluble and oligomeric forms of SOD-1 may reduce the availability of protein-folding chaperones that are required to catalyze the folding of other proteins. Direct evidence for the toxicity of aggregates or their misfolded intermediates comes from studies which show that both aggregate formation and toxicity can be reduced by the simultaneous expression of elevated levels of the protein-folding chaperone Hsp70 (Bruening et al., J. Neurochem. 72:693, 1999).

Many neurodegenerative diseases occur in a familial as well as a sporadic form, in which no mutation can be identified. In the case of ALS, the sporadic form accounts for the majority of cases. Indeed, SOD-1 aggregates have been identified in the spinal cords of individuals with sproradic ALS (Shibata et al., Neurosci. Lett. 179:149, 1994; Matsumoto et al., Clin. Neuropathol. 15:41, 1996). Furthermore, overexpression of wild type human SOD-1 in mice produces signs of toxicity and motor neuron dysfunction, and as well, increases or facilitates the progression of disease in SOD-1 mutant-expressing mice (Jaarsma et al., Neurobiol. Dis. 7:623, 2000). It therefore appears that ordered protein aggregation is a feature of both familial and sporadic forms of ALS, and further, that aggregation may be responsible for neurotoxicity.

At present there is no cure for ALS. Modalities for treating ALS presently being explored include: the use of neurotrophic factors to promote neuronal growth; glutamate uptake inhibitors to prevent uptake of toxic glutamic acid into neurons; free radical scavengers to prevent oxidative damage to proteins and DNA; and energy supplements such as creatine to increase metabolic energy. To date, none of the methods have shown any significant effect in altering the progression of ALS in affected individuals. One medication, RILUTEK, has been approved for marketing. The mode of action of this compound is unknown. It is clear that novel treatment modalities need to be investigated for use in altering ALS progression.

Accordingly, as abnormal protein conformation including misfolding and misassembly is linked to the progression of many diseases such as ALS, a need exists in the art for assays that identify agents that modulate protein aggregation or misfolding or that stabilize protein conformation, especially agents that inhibit, suppress, reduce, or attenuate protein aggregation, as well as agents that dissociate protein aggregates or reverse the aggregation process. The following invention addresses this need.

### SUMMARY OF THE INVENTION

As is disclosed herein, the inventors have designed a variety of in vitro and cell-based screening assays for identifying agents including human pharmaceuticals that prevent protein aggregation or induce stabilization of a native conformation of SOD-1 in vitro and in vivo. Once candidate agents are identified using such screens, cell-based and animal models are utilized to verify the effect of these agents in these systems. The assays disclosed herein are also readily applicable to any number of proteins that adopt an abnormal conformation including protein misfolding or protein aggregation that results in a pathological condition.

In general the invention features a variety of assays for identifying agents that modulate protein aggregation or stabilize protein conformation. A first screening assay involves in vitro aggregation and includes the steps of (i) combining protein molecules or fragments thereof and a candidate agent under conditions allowing for aggregation of the protein molecules; and (ii) determining whether aggregation of the protein molecules or fragments thereof are increased or decreased in comparison to aggregation of the protein molecules or fragments thereof in the absence of the candidate agent.

This assay is useful for the identification of agents that can modulate protein aggregation and can be applied to virtually any protein, which, when in an abnormal conformation including misfolding or aggregation, is known or believed to cause a conformational disease. In a preferred embodiment, the conformational disease is a neurological disease such as ALS. In another preferred embodiment, SOD-1, a protein whose abnormal conformation and aggregation contributes to the pathogenesis of ALS, is the protein used in the screening assay. The SOD protein can be any form of SOD including, but not limited to, mammalian SOD, SOD-1, human erythrocytic SOD-1, mutant SOD, or recombinantly produced SOD. SOD, if desired, may also be in the so-called apo, zinc (Zn)-deficient, as well as wild-type or mutant holoenzyme form of SOD.

For this in vitro aggregation assay, the methods used for determining protein aggregation can include any of the following: light scattering methodology, tryptophan fluorescence, UV absorption, turbidity measurement, a filter retardation assay, size exclusion chromatography, reversed-phase high performance liquid chromatography, an immunological assay, a fluorescent binding assay, a protein-staining assay, microscopy, or polyacrylamide gel electrophoresis (PAGE).

The preferred conditions for the in vitro aggregation assay include combining the protein and the agent in a metal-catalyzing oxidation buffer such as an ascorbate/copper (Cu) buffer for at least six hours at 37° C.

In another preferred embodiment of the in vitro aggregation assay, the assay is performed using wells of a microtiter plate to facilitate high-throughput robotics. High-throughput robotics is particularly useful when testing chemical agents or agents from chemical compound libraries.

The in vitro aggregation assay is useful for identifying an agent that either increases or decreases the aggregation of a protein as compared to the aggregation of the same protein in the absence of the agent. As increased protein aggregation is often linked to the pathogenesis of diseases, it is a preferred embodiment of this aspect of the invention that the agent identified decreases protein aggregation. Such agents are within the scope of this invention.

A second assay featured in the invention is a native state stabilization assay. This assay is used for identifying an agent that promotes a native conformation of a protein. This method includes the steps of (i) combining a protein and an agent under a condition that destabilizes the conformation of the protein and then (ii) determining whether the agent promotes the formation of a native conformation of the protein.

In another embodiment, the protein is a SOD protein such as mammalian SOD-1. Preferred forms of SOD-1 include apo-SOD-1, zinc-deficient SOD-1, or various mutant forms of SOD-1 as they are prone to destabilization under denaturation conditions including, but not limited to, thermally-induced or chemically-induced denaturation.

In the native state stabilization assay, the methods for determining protein aggregation can include any of the following: light scattering methodology, tryptophan fluorescence, UV absorption, turbidity measurement, a filter retardation assay, size exclusion chromatography, reversed-phase high performance liquid chromatography, an immunological assay, a fluorescent binding assay, a protein-staining assay, an assay for soluble protein, microscopy or polyacrylamide gel electrophoresis (PAGE).

The native state stabilization assay also includes methods for identifying an agent that promotes a native conformation of a SOD protein by determining whether the agent binds to SOD in its native state.

A third assay of the invention is a cell-based aggregation assay. This assay is useful for identifying an agent that modulates protein aggregation of a protein in a cell. This assay includes the steps of (i) providing a cell line which produces a protein and an agent under conditions allowing for aggregation of the protein in the cell line and then (ii) determining whether the aggregation of the protein in the cell line is increased or decreased in comparison to aggregation in the absence of the agent. In one preferred embodiments, the protein, such as SOD, results in a neurological disease, such as ALS, when it is expressed in its conformationally destabilized state in a human. Any form of SOD can be used; mammalian SOD-1 is preferred.

In a preferred embodiment of this method, the cell line is a mammalian cell line such as HEK293, COS, 3T3, or HeLa, that is used to overexpress SOD-1. In another preferred embodiment, the cell line is treated with a substance, such as a proteasome inhibitor (such as ALLN) that decreases the degradation of that protein. In this cell-based assay, aggregation is typically determined by immunological detection or a biochemical assay.

In a fourth assay an animal-based screen is used to identify an agent useful for treating a disorder resulting from expression of a conformationally destabilized protein. This method includes the steps of (i) administering a therapeutically effective amount of an agent identified in any of the above three assays to an animal that expresses a conformationally destabilized protein resulting in a conformational disease, and (ii) determining whether the agent decreases a disease symptom associated with expression of the conformationally destabilized protein, a decrease in the symptom as compared to control animals indicating that the agent is a useful pharmaceutical for treating the conformational disease.

In a preferred embodiment, the disorder is a neurological disease such as ALS. In another preferred embodiment of this aspect of the invention, SOD is the protein used in the assay. The SOD protein can be any form of SOD; mammalian SOD-1 is preferred. In another preferred embodiment, the animal used in the animal-based screen is a rodent or a transgenic rodent that overexpresses a protein such as SOD-1 or mutant forms of SOD-1.

In another aspect, the invention features a method of treating a human subject for a disease state associated with possession of a conformationally destabilized protein. This method includes the steps of administering to the human subject a therapeutically effective amount of one or more agents identified in any of the aforementioned screening assays. In a preferred embodiment, the disease is a neurological disease such as ALS.

By “aggregation of SOD-1” is meant a process whereby SOD-1 polypeptides associate with each other to form a multimeric, largely insoluble complex.

By “aggregation-prone intermediate” is meant a destabilized partially-folded form of a protein, which, under appropriate conditions, can aggregate or proceed to unfold to a more globally unfolded state.

By “amyloid protein” is meant a protein such as immunoglobulin light chains or amyloid protein A, that upon aggregation forms amyloid deposits, insoluble extracellular material of variable composition causing hardening, enlargement, and malfunction of an organ, tissue, or cell in which it is deposited.

By “apo-SOD-1” is meant SOD-1 that has no copper and no zinc atoms.

By “conformationally destabilized state” is meant the state of a protein resulting from a perturbation, alteration, or weakening of the interactions stabilizing native conformation of the protein.

By “conformational disease” is meant a disease for which aggregation of a protein into multimeric, largely insoluble complexes is symptomatic. Typically such protein aggregates are the causative agents of a pathology, and, as such, at least in part, the result of a gain of function. Such aggregates may form by self-association and deposition. In addition, the aggregates may consist, in part, of other proteins whose deposition is induced by a particular protein's self-association (e.g., the associated proteins found deposited with beta-amyloid protein in Alzheimer's disease).

By “holo SOD1” is meant SOD-1 that has its full complement of metals (i.e., two copper atoms and two zinc atoms per dimer).

By “inhibiting SOD-1 aggregation” is meant complete or partial inhibition of SOD-1 aggregation. Preferably, aggregation is inhibited at least 10%, more preferably, at least 20%, 30%, 40% or 50% or more. By “promoting SOD-1 aggregation” is meant an increase in the amount or rate or both of SOD-1 aggregation in the presence of the agent, as compared to the amount or rate or both of SOD-1 aggregation in the absence of the agent.

By “metal catalyzed oxidation buffer” is meant a buffer system that produces reactive oxygen species. Such a buffer typically contains a transition state metal and a reducing agent (such as an anti-oxidant).

By “native state” or “native conformation” is meant a naturally-occurring active conformation of a protein; such a conformation typically possesses appropriate elements of secondary and tertiary protein structure resulting in adoption of the naturally-occurring active structure.

By “polyglutamine protein” is meant a protein having one or more repeating regions of glutamines. Exemplary proteins include involucrin, huntingtin, ataxin-1, ataxin-2, ataxin-3, ataxin-7, alpha-1A voltage dependent calcium channel, androgen receptor, cystic fibrosis transmembrane conductance regulator, and atrophin-1.

By “protein” is meant any chain of amino acids, regardless of length or post-translational modification (for example, glycosylation or phosphorylation).

By “serpins” is meant a superfamily of serine protease inhibitors that share a complex, but well conserved, tertiary structure. Exemplary serpins include ovalbumin, the barley Z protease inhibitor, antitrypsin, and neuroserpin.

By “SOD-1 stabilizer” is meant an agent that binds to the native conformation of SOD-1 and by virtue of binding, stabilizes that native conformation.

By “zinc-deficient SOD-1” is meant SOD-1 that has its full complement of copper atoms (i.e., two copper atoms per dimer) but lacks its zinc atoms.

The invention represents an improvement over existing technology for identifying agents that modulate SOD-1 aggregation in several ways. For example, the present invention provides agents that affect the aggregation of SOD-1 and therefore can be used to treat subjects having a disorder associated with aberrant SOD-1 aggregation, e.g. ALS. The aggregation and deposition of SOD-1 plays an important role in the pathology of the disease. Thus, modulators or stabilizers identified using the methods and assays described herein can affect aggregation of SOD-1 and are therefore suitable for therapeutic use in vivo. Additionally, the methods disclosed herein provide sensitive detection methods that retain samples under native or physiological conditions which are especially useful for identifying SOD-1 aggregation modulators using high throughput screening methods. Accordingly, the methods and assays described herein are of immediate value for their ability to identify agents (e.g., organic or inorganic compounds) for pharmaceutical or other applications in treating diseases typified by SOD-1 aggregation such as ALS.

Other features and advantages of the invention will be apparent from the following description of the preferred embodiments thereof, and from the claims.

### BRIEF DESCRIPTION OF THE DRAWINGS

FIG. 1 shows the structure of human SOD-1. Molecular modeling of human SOD-1 was carried out using the program InsightII (Accelrys, Burlington, Mass.) using protein data bank (pdb) coordinates from 1SPD. FIG. 1A shows that SOD-1 is a homodimeric enzyme responsible for the redox-catalyzed detoxification of superoxide. SOD-1 exists primarily in a β-barrel conformation with each subunit containing one Cu and one Zn atom. FIG. 1B illustrates a 90° rotation of the view shown in FIG. 1A showing the detail of the active site Cu and Zn atoms in one SOD-1 monomer. The Cu is coordinated by 4 histidine residues and is necessary for the redox activity of SOD-1. The Zn atom is coordinated by 3 histidine residues and one aspartic acid residue and is required for maintaining the shape of the active site of SOD-1 but not required for SOD-1 activity.

FIG. 2 shows a schematic representation of two different methods that can be used to screen for agents capable of modulating or stabilizing SOD-1 protein conformation in vitro. FIG. 2A shows that upon application of stress (e.g., thermal or oxidative stress) a slightly unfolded aggregation-prone intermediate becomes populated. In the absence of any stabilizing factors, the intermediate goes on to form aggregates. FIG. 2B shows that one method to prevent aggregation of SOD-1 is to identify agents which bind to the aggregation-prone intermediate or to the aggregate and result in blocking sites that may be responsible for association of these species. FIG. 2C shows a second method of screening for inhibitors that relies on identifying agents that bind to SOD-1.

FIG. 3 shows the metal catalyzed oxidation (MCO)-induced aggregation of SOD-1. FIG. 3A shows the detection of MCO-induced SOD-1 aggregation by right angle light scattering (RALS) measurements. FIG. 3B shows the detection of MCO-induced SOD-1 aggregation before and after a 37° C. incubation period by RALS. RALS of SOD-1 aggregates was measured using a DynaPro99-MSXTC/12 instrument. FIG. 3C shows the detection of MCO-induced SOD-1 aggregation by dynamic light scattering (DLS).

FIG. 4 shows the detection of SOD-1 aggregates using different biophysical methods. FIG. 4A shows detection of SOD-1 aggregates by UV absorption. (Sample pH was as follows: A, 4.97; B, 5.37; C, 5.83; D, 6.05; E, 6.17; F, 6.44; G, 6.64; H, 6.75; I, 7.01; and J, 7.19.) FIG. 4B shows detection of SOD-1 aggregates by RALS. FIG. 4C shows detection of SOD-1 aggregates using tryptophan (Trp) fluorescence.

FIG. 5 shows the detection of SOD-1 aggregates using electron microscopy and atomic force microscopy. FIGS. 5A and 5B show detection of SOD-1 aggregates by negative stain electron microscopy. The magnification is 25,000 and 57,000 in FIGS. 5A and 5B, respectively. FIG. 5C shows the detection of SOD-1 aggregates by atomic force measurement microscopy.

FIG. 6 shows MCO-induced aggregation of zinc-deficient and mutant SOD-1 as detected by RALS. FIG. 6A shows wild-type holoenzyme, zinc-deficient SOD-1, or mutant holoenzyme SOD-1 at a concentration of 10 μM SOD-1 incubated in the presence of 4 mM ascorbic acid and 0.2 mM CuCl₂ in 10 mM Tris, 10 mM acetate buffer, pH 7.0 (black bars) whereas control reactions were 10 μM SOD-1 in buffer (gray bars); reactions were incubated at 37° C. for 48 hours. FIG. 6B shows MCO-induced SOD-1 aggregation is pH dependent.

FIG. 7 shows MCO-induced modifications of SOD-1. Human wt SOD-1 at a concentration of 30 μM in 10 mM sodium acetate buffer, pH 5.0 was incubated in the presence of 2 mM ascorbate and 25 μM copper at 60° C. and aliquots of supernatants analyzed at the times indicated by SDS PAGE (FIG. 7A) or native PAGE (FIG. 7B). Pellets were also analyzed by native PAGE (FIG. 7B) after a 24 hour incubation by centrifuging incubated samples and suspending pellets in sample buffer. Lanes labeled “C” represent control samples incubated under the same conditions in the absence of copper and ascorbate. The bracket indicates the bands on the gel that are indicative of conformational heterogeneity.

FIG. 8 shows the detection of SOD-1 aggregation using biochemical methods. FIG. 8A shows an analysis of MCO-induced SOD-1 aggregation by sodium dodecyl sulphate polyacrylamide gel electrophoresis (SDS PAGE). FIG. 8B shows an analysis of MCO-induced SOD-1 aggregation by native PAGE. FIG. 8C shows an analysis of MCO-induced SOD-1 aggregation by filter retardation assay. FIG. 8D shows an analysis of MCO-induced SOD-1 aggregation by size exclusion chromatography (SEC). FIG. 8E shows an analysis of MCO-induced SOD-1 aggregation by filter retardation assay after dissolution of the insoluble aggregates in a 1% SDS solution.

FIG. 9 shows an analysis of MCO-induced SOD-1 aggregation by SEC. FIG. 9A shows a time course for SOD-1 aggregation as measured by monitoring the amount of soluble SOD-1 remaining in solution following treatment of human wt SOD-1 at a concentration of 30 μM in 10 mM sodium acetate buffer, pH 5.0 with 2 mM ascorbate and 25 μM copper at 37° C. Aliquots of supernatant were analyzed by SEC at the times indicated. FIG. 9B shows the temperature dependence of SOD-1 aggregation. SOD-1 aggregation was determined as in FIG. 9A by incubating SOD-1 treated with Cu and ascorbate at different temperatures and comparing peak areas to those of controls to determine the amount of soluble SOD-1 remaining. FIG. 9C shows the pH dependence of aggregation. SOD-1 aggregation was determined as in FIG. 9A with the exception that Cu and ascorbate treatment were carried out at 60° C. in 10 mM Tris-acetate buffer at the pH values indicated after 24 hours.

FIG. 10 shows a competitive ELISA assay used to detect SOD-1 aggregation. FIG. 10A shows a schematic of the methodology used to carry out a competitive ELISA assay to measure the amount of aggregated SOD-1 following treatment using a MCO system. FIG. 10B shows an example of a competitive ELISA.

FIG. 11 shows two independent examples of a competitive ELISA using SOD-1 or MCO-treated SOD-1.

FIG. 12 shows the results from an amino acid analysis of untreated and MCO-treated SOD-1.

FIG. 13 shows the results from mass spectrometric analysis of tryptic peptides derived from untreated and MCO-treated SOD-1.

FIG. 14 shows molecular modeling of MCO-treated human SOD-1. Molecular modeling was carried out using the program InsightII (Accelrys, Burlington, Mass.) using pdb coordinates from 1SPD. The oxidized sites present in SOD-1 treated with Cu and ascorbate as determined by mass spectroscopic analysis are mapped onto the structure of a monomer of SOD-1 and shown as either a side view (FIG. 14A) or top view (FIG. 14B). Shown also are copper and zinc atoms in the active site.

FIG. 15 shows the inhibition of MCO-induced SOD-1 aggregation using EDTA and anaerobic conditions.

FIG. 16 shows an ANS dye binding assay to characterize the folded state of SOD-1 under various conditions. FIG. 16A shows the results from ANS dye binding assays. Human wt SOD-1 at a concentration of 3 μM incubated in the presence of 4 mM ascorbate and 200 μM copper in 10 mM sodium acetate buffer, pH 5.0, at 60° C. for 29 hours. Control preparations were incubated in the absence of copper and ascorbate under the same conditions. Following incubation, samples were vortexed thoroughly and ANS dye contained in DMSO added to a final concentration of 20 μM ANS. Samples were incubated at room temperature for 20 minutes and fluorescence measured on a Cary-Varian Eclipse Spectrofluorimeter using an excitation of 372 nm and emission recorded between 400-600 nm. FIG. 16B shows results from ANS dye binding assays. Apo-SOD-1 at a concentration of 30 μM in 10 mM sodium acetate buffer, pH 5.0 was incubated at either 60° C. or 4° C. ANS dye binding was carried out as described in FIG. 16A.

FIG. 17 shows a native state stabilization assay using apo-SOD-1. FIG. 17A shows results where aliquots of supernatants were analyzed by reversed-phase high performance liquid chromatography (RP HPLC) and quantitation of amounts of soluble SOD-1 remaining following incubation. FIG. 17B shows detection of SOD-1 aggregates by protein staining technique. Supernatants were removed and tubes washed 4 times with 10 mM sodium acetate buffer, pH 5.0, and washes discarded. To the tubes was added 100 μl of micro BCA protein determination reagent (Pierce), the tubes sealed and incubated at 60° C. for up to 1 hour to allow for color development. The amount of protein present was quantitated by measuring absorbance at 562 nm.

FIG. 18 shows a native state stabilization assay used to test various agents for inhibition of aggregation of apo-SOD-1. FIG. 18A shows analysis of SOD-1 aggregation by RP-HPLC. FIG. 18B shows a graph depicting peak areas from chromatograms derived in FIG. 18A that were derived and plotted to show amount of soluble SOD-1 remaining after each treatment.

FIG. 19 shows immunocytochemical staining of HEK293A cells transfected with SOD-1. HEK293A cells were transfected with HA-tagged human wt SOD-1 (FIG. 19A), HA-tagged human mutant G85R SOD-1 (FIG. 19B), or HA-tagged human mutant G41S SOD-1 (FIG. 19C).

FIG. 20 shows biochemical assays for cell based aggregation. HEK293A cells were transfected with human wt SOD-1 or human mutant SOD-1 cDNA contained in the pFLUC plasmid (Valentis) and analyzed by SDS PAGE/Western blot analysis under reducing conditions (FIGS. 20A and 20B) or native PAGE/Western blot analysis (FIGS. 20C and 20D).

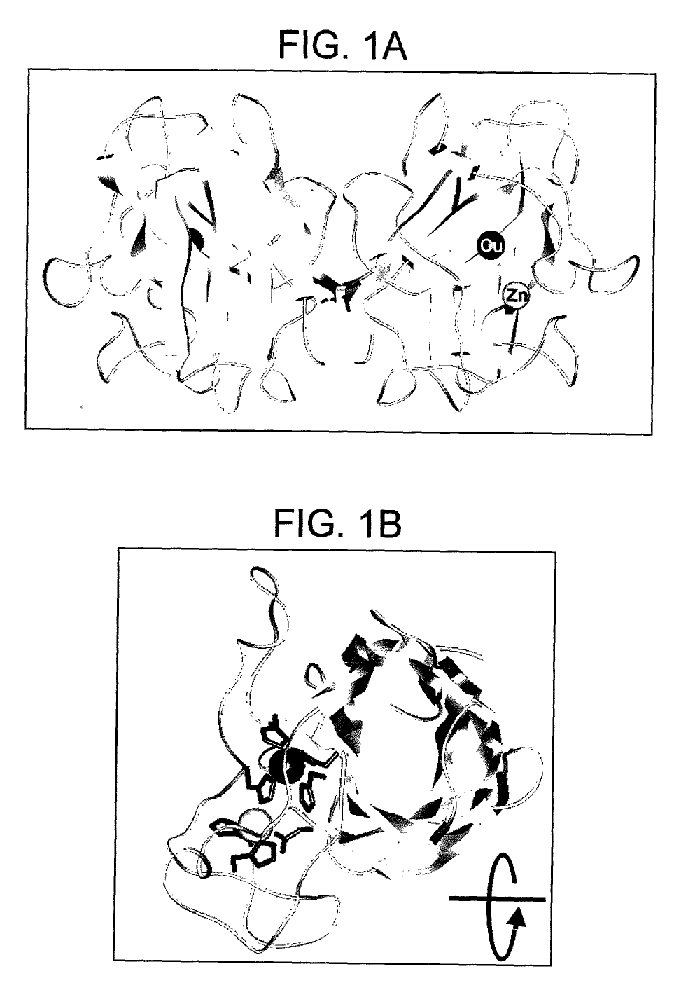

### DESCRIPTION OF THE INVENTION

Below methods and assays are described for inducing and detecting protein aggregation in vitro and in cell-based assays and animal models. These methods can be used to identify molecules that modulate (for example, agonize or antagonize) protein aggregation, which may, in turn, be useful for treatment of diseases associated with abnormal protein conformation. The disclosed methods are also useful for identifying agents that stabilize the native conformation of proteins. Methods for screening candidate agents for dissociating protein aggregates and for reversing the aggregation process are also disclosed.

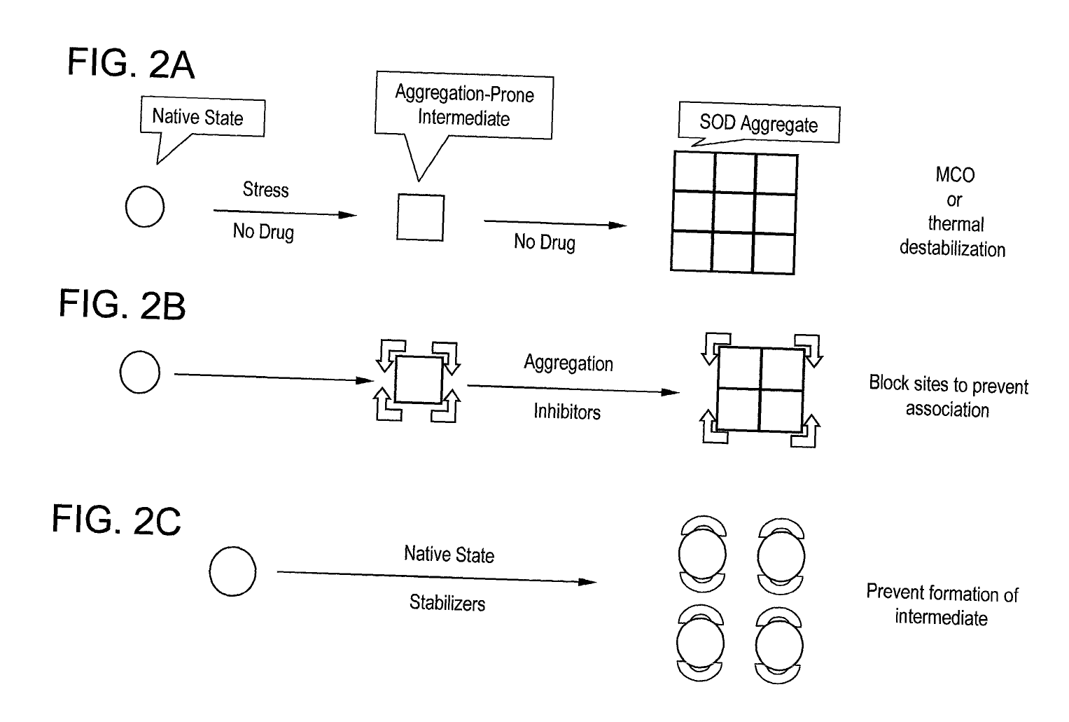

The present invention features, in general, four categories of methods for identifying agents that modulate protein aggregation. The first category includes in vitro aggregation assays. The in vitro aggregation assays are used, for example, to measure metal catalyzed oxidation (MCO) induced aggregation of a protein such as SOD-1 and then to screen for agents that reduce or prevent this aggregation. One method to identify potential protein aggregation inhibitors is to identify agents which bind to an aggregation-prone intermediate or to the aggregate and result in blocking sites that may be responsible for association of these species. It is likely that such an inhibitor will bind to hydrophobic sites since it has generally been found that when proteins aggregate hydrophobic residues are exposed as a result of local or more global unfolding; these hydrophobic groups are normally present in the interior of the folded protein and are typically shielded from water (FIG. 2B).

A second category of methods for identifying agents that modulate protein aggregation includes native state stabilization assays. Native state stabilization assays complement the above-mentioned in vitro aggregation assays. Native stabilization assays, however, are not generally based on MCO-induced aggregation but rather on the propensity of a destabilized conformation of a protein to form aggregates. For the native state stabilization assays, destabilization occurs through the use of a variety of protein denaturation techniques, such as application of heat, chemicals, or through the use of specific forms of the protein that are more prone to conformational destabilization such as apo-SOD-1, zinc-deficient SOD-1 or mutant SOD-1.

Native state stabilization assays are then used to screen for agents which stabilize the native state, preventing destabilization and aggregation. It is generally seen that if an agent binds the native state of a protein, the binding results in stabilization of that native state. For example, if an agent binds to SOD-1, it will stabilize the native folded state of SOD-1 and prevent or limit the formation of the aggregation-prone intermediate and hence aggregates upon addition of a stress (FIG. 2C). One can readily screen for such native state stabilizers, for example, aggregation inhibitors, by at least two methods. First, by screening for agents using a standard binding assay to identify agents that interact with SOD-1. Exemplary binding assays include, without limitation, Biacore measurements in which a potential ligand is immobilized and a SOD solution passed over the bound ligand and binding measured; binding of radio-, fluorescently- or biotin-labeled compounds to immobilized SOD; or by immobilizing a ligand and identifying binding of a detectably-labeled SOD molecule (for example, a SOD molecule chemically labeled (e.g., with biotin, or a fluorescent tag), or SOD immunologically-detected. Other assays for assessing binding of an agent to SOD include, but are not limited to, standard ligand blotting assays, assays of enzyme activity, protein gel-shift assays, spectroscopy including NMR and CD spectroscopy, differential scanning calorimetry, monitoring susceptibility to proteolytic digestion, and LC/MS measurements to monitor and identify ligand binding. In addition, a mass-encoded library approach can be used to identify agents that bind to SOD. Mass-encoded libraries contain a set of small molecules that are individually distinguishable by their mass, thus, for example, upon release from their bound state, mass spectroscopy can be performed to definitively identify which small molecules bound to a particular target. A second screening approach involves identifying agents that affect protein aggregation resulting from a destabilization stress (such as a temperature- or chemical-induced denaturation).

A third category of methods for identifying agents that modulate protein aggregation includes cell-based aggregation assays. These cell-based assays measure protein aggregation in an in vivo system and are particularly useful as a secondary screen for potential agents that modulate protein conformation and aggregation. Agents that are identified in either of the above categories can then be tested in these in vivo assays to measure the ability of the agent to modulate protein conformation and aggregation in a more biologically relevant setting.

A fourth category of methods includes testing agents identified in any of the aforementioned screens in animal models to determine the effect of those agents in a disease system.

The following detailed description uses ALS and SOD-1 as a specific example of a disease (ALS) involving abnormal protein conformation and aggregation (SOD-1); however, it will be appreciated by any person skilled in the art that any of the methods described herein can be used to identify agents and devise treatments for other diseases that involve the inappropriate aggregation or destabilization of a protein with only minor modifications. The methods of the present invention would preferably be used to identify agents and devise treatments for conformational diseases, more preferably for neurodegenerative diseases, or diseases attributed to aggregating poly-glutamine containing, polyalanine-containing, serpin, or amyloid proteins. Most preferably the methods would be used to identify agents and devise treatments for ALS. Examples of conformational diseases and relevant proteins include (each disease-protein combination is written as disease (protein)) neurodegenerative diseases such as ALS (SOD-1); Huntington's disease (Huntingtin); Parkinsons' disease (alpha-synuclein); Alzheimer's disease (beta-amyloid peptide); Creutzfeldt-Jakob disease (prion); Pick's disease (tau); cystic fibrosis (cystic fibrosis transmembrane conductance regulator); spinocerebellar ataxia 1 (ataxin-1); spinocerebellar ataxia 2 (ataxin-2); spinocerebellar ataxia 3/Machado-Joseph disease (ataxin-3); spinocerebellar ataxia 6 (alpha-1A voltage dependent calcium channel); spinocerebellar ataxia 7 (ataxin-7); spinobulbar muscular atrophy/Kennedy disease (androgen receptor); denatorubro-pallidoluysian atrophy/Haw River Syndrome (atrophin-1); cirrhosis (antitrypsin); emphysema (antitrypsin); hereditary cardiac amyloidosis (apolipoprotein A-I); Finnish type familial amyloidosis (gelsolin); familial amyloid polyneuropathy, familial amyloid cardiomyopathy, and senile system amyloidosis (transthyretin); senescence (lactadherin); familial dementia (neuroserpin); cataracts (alpha-crystallin); Type II diabetes (islet amyloid polypeptide); retinitis pigmentosa (rhodopsin); immunoglobulin amyloidosis (immunoglobulin light chain, gamma 1 heaavy chain); white sponge naevus (keratin); chronic inflammatory disease (serum amyloid A); systemic amyloidosis-ALys (lysozyme); systemic amyloidosis-AFib (fibrinogen A alpha); systemic amyloidosis secondary (amyloid A protein); dialysis-associated amyloidosis (beta-2 microglobulin); senile cardiac atria amyloidosis (atrial natriuretic factor); medullary carcinoma thyroid endocrine amyloidosis (procalcitonin); systemic vascular amyloidosis HCHWA (cystatin C); or any disease involving the inappropriate aggregation or destabilization of polyglutamine, polyalanine, amyloid or serpin family proteins.

Superoxide Dismutase (SOD)

Superoxide dismutases (SOD) are metalloenzymes that catalyze the destruction/dismutation of superoxide free radical ions into oxygen and hydrogen peroxide. Three classes of SOD have been described in the literature, each characterized by the presence of a catalytic metal at the active site of the enzyme: Cu/Zn-SOD-1 (SOD-1), Mn-SOD, and Fe-SOD. While these different SODs carry out the same dismutation reaction, they are structurally and spatially distinct. SOD-1 is found primarily in the cytoplasm and, to date, is the only SOD-1 in which aggregation has been implicated in the progression of ALS. These SODs are widely distributed in nature and are readily isolated and purified from a variety of organisms such as bacteria, plants, fungi such as yeast, amphibians, and mammals such as humans and bovines. For example, in humans, SOD-1 is present in high concentrations in brain, liver, heart, erythrocytes, and kidney, and is readily purified and isolated using standard methods.

Accordingly, the skilled worker will understand that SODs from virtually any source may be used in a variety of the methods or assays disclosed herein. In preferred embodiments, human SOD-1 is utilized in the methods and assays described herein. The naturally occurring human SOD-1 polypeptide has a length of 153 amino acids and is highly homologous (>70%) with the SOD-1 polypeptides expressed in other vertebrates. In other embodiments, the methods and assays employ mutant SOD-1 polypeptides such as a FALS-associated SOD-1 mutant. More than 63 different mutations at 43 codons of such FALS-associated mutants have been described to date (see, for example, Orrell, Neuromuscular Disorders, 10:63, 2000). Some well-known SOD-1 mutations useful in the methods of the invention include A4V, D90A (Cleveland and Rothstein, Nature Neurosci. 2:806, 2001), G93A, D124N (Banci et al., Eur. J. Biochem. 196:123, 1991), A4T (Takahashi, H. et al., Acta Neuropathol. 88:185, 1994), G37R (Cudkowicz, M. E. et al., Ann. Neurol. 41:210, 1997), and G85R (Deng, H. X. et al., Science 261:1047, 1993). In other preferred embodiments, any additional forms of SOD can be used including, but not limited to, bovine, equine, porcine, or rat SOD, or SOD-1 human homologs.

In addition to full-length, naturally occurring SOD-1 polypeptides and SOD-1 mutant polypeptides, the assays described herein may also employ SOD-1 fragments of such polypeptides. SOD-1 fragments may range in size from five amino acid residues to the entire amino acid sequence of the SOD-1 molecule minus one amino acid. In preferred embodiments, a peptide fragment of SOD-1 includes at least 10 contiguous amino acids, preferably at least 20 contiguous amino acids, more preferably at least 30 contiguous amino acids, and most preferably at least 40 to 50 or more contiguous amino acids of a SOD-1 polypeptide. Fragments of SOD-1 polypeptides can be generated by methods known to those skilled in the art (e.g., chemical synthesis) or may result from normal protein processing (e.g., removal of amino acids from the nascent polypeptide that are not required for biological activity or removal of amino acids by alternative mRNA splicing or alternative protein processing events).

The invention further includes aggregation assays that make use of analogs of any naturally occurring SOD-1. Analogs can differ from the naturally occurring or mutant SOD-1 by amino acid sequence differences, by post-translational modifications, or by both. In preferred embodiments, SOD-1 analogs used in the invention will generally exhibit about 30%, more preferably 50%, and most preferably 60% or even having 70%, 80%, or 90% identity with all or part of a naturally-occurring a SOD-1 amino acid sequence e.g., the human SOD-1 amino acid sequence. The length of sequence comparison is at least 10 to 15 amino acid residues, preferably at least 25 amino acid residues, and more preferably more than 35 amino acid residues. Modifications include chemical derivatization of polypeptides, e.g., acetylation, carboxylation, phosphorylation, or glycosylation; such modifications may occur during polypeptide synthesis or processing or following treatment with isolated modifying enzymes.

SOD-1 analogs can also differ from the naturally occurring polypeptides by alterations in primary sequence. These include genetic variants, both natural and induced; for example, those polypeptides resulting from random mutagenesis by irradiation or exposure to ethyl methylsulfate or by site-specific mutagenesis as described, for example, in Sambrook, Fritsch and Maniatis (Molecular Cloning: A Laboratory Manual (2d ed.), CSH Press, 1989) or in Ausubel et al. (Current Protocols in Molecular Biology, John Wiley and Sons, 2000).

Also included are cyclized SOD-1 peptides, molecules, and analogs which contain residues other than L-amino acids, e.g., D-amino acids or non-naturally occurring or synthetic amino acids, e.g., β or γ amino acids. For example, a SOD-1 polypeptide used in the assays disclosed herein may have an amino acid sequence that is identical to that of the naturally-occurring SOD-1 polypeptide or that is different by minor variations due to one or more amino acid substitutions. The variation may be a “conservative change” typically in the range of about 1 to 5 amino acids, wherein the substituted amino acid has similar structural or chemical properties, e.g., replacement of leucine with isoleucine or threonine with serine. In contrast, variations may include nonconservative changes, e.g., replacement of a glycine with a tryptophan. Similar minor variations may also include amino acid deletions or insertions, or both. Guidance in determining which and how many amino acid residues may be substituted, inserted or deleted without changing biological activity may be found using computer programs well known in the art, for example, DNASTAR software (DNASTAR Inc., Madison Wis.). Fragments, analogs, variants, and mutants of SOD-1 preferably retain the ability to aggregate in any of the assays described herein.

Any of the aforementioned SOD-1 polypeptides are prepared according to standard methods known in the art. For example, SOD-1 polypeptides may be prepared by standard chemical peptide synthesis techniques. SOD-1 may also be purchased from any number of commercial suppliers including Sigma-Aldrich Fine Chemicals (St. Louis, Mo.), Research Diagnostics Inc, Flanders, N.J., and Calbiochem-Novabiochem Corporation, LaJolla, Calif.

Alternatively, a SOD-1 polypeptide may be prepared using recombinant methods. Generally this involves creating a DNA sequence that encodes the SOD-1 polypeptide, placing the DNA in an expression cassette under the control of a particular promoter, expressing the SOD-1 polypeptide in a host, isolating the expressed SOD-1 polypeptide and, if required, renaturing the polypeptide.

The nucleic acid sequences encoding the SOD-1 polypeptide can be expressed in a variety of host cells, including bacteria, yeast, insect cells, mammalian cells, or plant cells. In preferred embodiments, SOD-1 is produced in bacterial or insect cells. The recombinant SOD-1 gene, in general, is operably linked to appropriate expression control sequences for each host. By “operably linked” is meant that a gene encoding a SOD-1 polypeptide and a regulatory sequence(s) are connected in such a way as to permit gene expression when the appropriate molecules (for example, transcriptional activator proteins) are bound to the regulatory sequence(s). For E. coli this includes a promoter such as the T7, trp, or lambda promoters, a ribosome binding site, and preferably a transcription termination signal. For eukaryotic cells, the control sequences will include a promoter and preferably an enhancer derived from immunoglobulin genes, SV40, or cytomegalovirus, and a polyadenylation sequence, and may include splice donor and acceptor sequences. Exemplary SOD-1 polypeptides produced using recombinant techniques have been described by Crow et al. (J. Neurochem. 69:1936, 1997) and Fujii et al. (J. Neurochem. 64:1456, 1995).

Once expressed, the SOD-1 polypeptide can be purified according to standard procedures known in the art, including ammonium sulfate precipitation, affinity columns, column chromatography, and gel electrophoresis (see, generally, Michalski, J. Chromatog. B684:59, 1996). Substantially pure compositions of at least about 90 to 95% homogeneity are preferred, and 98 to 99% or more homogeneity are most preferred. Once purified, partially or to homogeneity as desired, the polypeptides are then be used in the methods or assays described herein.

SOD-1 Aggregation

SOD-1 normally is a highly stable protein with a Tm of approximately 80° C. for the fully metallated form (Rodriguez et al., J. Biol. Chem., 277:15923, 2002). Upon application of stress, for e.g. such as thermal or oxidative stress, a slightly unfolded aggregation-prone intermediate becomes populated. In the absence of any stabilizing factors, the intermediate goes on to form aggregates (FIG. 2A).

It is known that under appropriate conditions SOD-1 can catalyze a reverse redox reaction in which the normal enzymatic product, hydrogen peroxide, is converted to superoxide and hydroxyl free radical:

2H₂O₂→O₂⁻+2H⁺+OH^{-+OH}^{·}

In the absence of suitable substrates for oxidation, SOD-1 itself is oxidized and inactivated by the hydroxyl free radical (see Goto et al., J. Biol. Chem., 273:30104, 1998 and references therein). We mimic this oxidation of SOD-1 through a metal-catalyzed oxidation (MCO) system employing copper and ascorbate as the reactive oxygen species generation system.

Treatment of SOD-1 with copper and ascorbate results in the following process: chemical modifications of SOD-1, conformational changes to SOD-1 followed by unfolding and aggregation of SOD-1.

To mimic in vitro the misfolding and aggregation of SOD-1 observed in vivo, a purified SOD-1 polypeptide is typically incubated in a solution that promotes its aggregation. In preferred embodiments, the incubation solution used to aggregate SOD-1 in vitro is a solution, preferably a buffered solution, that generates reactive oxygen species, such as H₂O₂, O₂⁻, and HO^{·} that oxidizes susceptible chemical groups in proteins. Exemplary solutions useful for generating such reactive oxygen species include, without limitation, ascorbic acid or hydrogen peroxide. In preferred embodiments, a buffered ascorbate solution is utilized, and the concentration of ascorbic acid used to induce aggregation is within the normal physiological range of ascorbic acid concentrations found in neurons which can be as high as 10 mM (Rice, Trends In Neurobiology 23:209, 2000).

In other preferred embodiments, a transition metal-catalyzed (e.g., Fe^{2+/3+}, Cu²⁺, Cu¹⁺, Hg^{1+/2+}, Pb^{2+/3+}, Sn^{2+/4+}, MnO₄, MnO₃⁻, Cr₂O₇, and CrO₄) oxidizing buffer such as a Cu²⁺-ascorbate buffer as disclosed herein is utilized. Without being bound by theory, SOD-1 aggregation, under such metal catalyzed oxidation conditions, is induced by a metal-catalyzed oxidation (MCO) reaction according to the following scenario. Ascorbic acid reduces the bound Cu²⁺ ion of SOD-1 to Cu¹⁺. The bound Cu¹⁺ reacts with H₂O and O₂ to produce H₂O₂, O₂⁻, and HO^{·}, these reactive oxygen species then oxidize susceptible chemical groups in SOD-1. In particular, the oxidation reactions are thought to induce a structural change in SOD-1 resulting in the formation of an aggregation-prone conformation. Addition of exogenous Cu²⁺ results in increased generation of reactive oxygen species, thereby accelerating SOD-1 oxidation and aggregation.

Additionally, if desired, a reactive oxygen generating buffer system may be utilized. Such a buffer system includes H₂O₂ at a concentration of 10 μM-1 mM.

In additional preferred embodiments a native state stabilization assay can be used to identify agents that bind to, and stabilize the native state of SOD-1. Alternatively, agents that bind to SOD-1 can be identified using many methods. These can then be screened for their ability to stabilize the native conformation. The native state stabilization assay is based on the ability of destabilized SOD to unfold and aggregate. In this assay, apo-SOD-1, zinc deficient SOD-1 or mutant forms of SOD-1 known to unfold and aggregate under conditions of thermal or chemical stress is preferred. For example, temperatures which induce destabilization of various forms of SOD-1 are as follows: apo-SOD-1 at 50°-60° C.; zinc-deficient SOD-1 at 60°-70° C.; wild type SOD-1 at 70°-80° C.; and mutant SOD-1 at 60°-80° C. Additional temperature ranges for each of the aforementioned forms are SOD are determined according to standard methods as is described herein.

Examples of chemicals which induce destabilization include, without limitation, guanidine thiocyanate and organic solvents (methanol, ethanol, n-propanol, isopropanol, dimethylformamide, dimethylsulfoxide).

SOD-1 is included in the aggregation solution at a concentration of at least 1 μM, preferably at 10 μM, and more preferably at 10-50 μM.

The pH of the aggregation solution is at least 5, preferably between 5.5 and 7, and more preferably between 5.8 and 7. The assay incubation solution can also include a variety of other reagents, such as salts, buffers, organic solvents, organic solutes, or additional proteins.

The assay mixtures are incubated under conditions in which SOD-1 polypeptides aggregate, if not for the presence of the potential aggregation modulator agent or an agent that promotes reversal of aggregation. The solution mixture components can be added in any order that provides for the requisite aggregation. Incubations may be performed at any temperature which facilitates optimal aggregation, typically between 20° and 60° C., depending on the type of assay used. Incubation periods are likewise selected for optimal aggregation but are also minimized to facilitate rapid, high-throughput screening, and are typically between 0 and 96 hours, preferably less than 48 hours, more preferably less than 24 hours. For optimal high throughput applications, the reaction is carried out for between 1 and 96 hours, more typically between about 12 and 48 hours.

After incubation, SOD-1 aggregation is detected by any of the methods described below.

SOD-1 Detection

SOD aggregation is monitored either directly, for example, by detecting aggregated SOD or indirectly by measuring the loss of soluble SOD.

Exemplary direct methods for detecting SOD-1 aggregation in vitro include, without limitation, optical techniques such as right angle light scattering (RALS), dynamic light scattering (DLS), UV fluorescence/turbidity, and tryptophan (Trp) fluorescence analysis; microscopic techniques such as electron microscopic imaging (EM) and atomic force microscopy imaging (AFM); chromatographic techniques such as size exclusion chromatography (SEC) and reversed-phase-HPLC (RP-HPLC) (see, for example, “Protein Purification: Principles and Practice”, R. K. Scopes, ed., Springer-Verlag, New York; 1987); and biochemical techniques such as fluorescent staining using ANS or bis-ANS (e.g., ANS dye binding assays as described in Stryer, J. Mol. Biol. 13:482, 1965), polyacrylamide gel electrophoresis (PAGE), and filter retardation assays; and immunological methods (e.g., ELISA).

In particular, RALS relies on the ability of protein aggregates to scatter light (see, for example, Classical Light Scattering from Polymer Solutions, P. Kratochvil, Elsevier, Amsterdam, 1987), and light scattering measurements can be made using a standard fluorometer. Standard methods for obtaining aggregation data of a protein using RALS are known in the art and are described, for example, in “Classical Light Scattering from Polymer Solutions,” supra.

Another optical technique, DLS, measures fluctuations of the scattered light intensity of an aggregate as a function of time. An autocorrelation function is used to evaluate the fluctuations in the intensity of the scattered light which, in turn, is used to calculate the diffusion coefficient of particles in the sample that cause the light scattering. A regularization algorithm is then used to estimate how many different species of scattering particles should be included in the data analysis. Standard methods describing DLS are found in “Dynamic Light Scattering: Applications of Photon Correlation Spectroscopy,” Pecora, R., ed., Plenum Press, 1985.

In addition, UV absorption methodologies are useful for detecting the presence of aggregates in solution. The principle of this absorption method is that the presence of aggregates increases the turbidity of the solution and, therefore, increases the apparent absorbance. Maintenance of identical concentrations of protein and buffer components in all samples ensures that any increase in absorbance of the sample is attributable to the presence of aggregates. For these measurements, UV light is preferred over visible light because the apparent absorbance caused by the presence of aggregates increases at lower wavelengths. It should be noted however that small particles or aggregates which adsorb to the sides of the incubation vessel will not be seen by this method. Exemplary methods describing the uses of UV/turbidity analysis for aggregate detection are found in “Physical Biochemistry: Application to Biochemistry and Molecular Biology,” D. Freifelder, ed., W. H. Freeman and Company, San Francisco, 1982, p. 504 and “Biophysical Chemistry Part II: Techniques for the Study of Biological Structure and Function,” Cantor, C. and Schimmel, P., eds., W. H. Freeman and Co, New York; 1980.

In yet another method for detecting SOD-1 aggregation in vitro, Trp fluorescence measurements are performed on an incubated sample to determine whether metal-catalyzed oxidation induced structural changes in SOD-1. In its three-dimensional conformation, SOD-1 possesses a single Trp residue exposed to solvent. Since the aggregation process will change the chemical environment of the Trp residue, the environmental change may alter fluorescent properties of Trp such as the quantum yield. Methods for determining protein aggregation using UV/turbidity measurements are described in “Biophysical Chemistry Part II: Techniques for the Study of Biological Structure and Function,” supra.

SOD-1 aggregates can also be detected utilizing a standard filter retardation assay. In this detection methodology, solutions suspected to contain SOD-1 aggregates are passed through membranes such as nitrocellulose, cellulose acetate, and polyvinylidene fluoride, and aggregates present in the solution are trapped by the membrane. The immobilized aggregates are then detected by any standard detection method such as immunostaining. Aggregates present in the insoluble material can also be detected using a filter retardation assay by dissolving the pellet in an SDS solution prior to membrane filtration.

The presence of SOD-1 aggregates may also be analyzed using standard methods of electron microscopy. For example, negatively stained SOD-1 aggregates are prepared by floating charged pioloform, carbon-coated grids on aggregated SOD-1 solutions. The grids are then blotted and air-dried, and stained, for example, with 1% (w/v) phosphotungstic acid. Representative electron microscopy images of the SOD-1 aggregates are then obtained using standard methods. Atomic force microscopy can also be used to analyze SOD-1 aggregates. For AFM measurements, images are obtained using a Digital Instruments NanoScope III® atomic force microscope. Samples were deposited onto freshly cleaved mica and dried under positive pressure. Contact mode images were obtained using a Si₃N₄ tip (Digital Instruments) with spring constant of 0.12 N/m.

Additional biochemical methods that can be used to detect SOD-1 aggregation include PAGE and immunological methods such as ELISA. Detection of SOD aggregation by PAGE includes both denaturing conditions (SDS PAGE) and non-denaturing conditions (native PAGE). ELISA techniques include three assays: direct, sandwich, and competitive assays.

For direct ELISA assays, SOD-1 (standard curve and supernatants from control or aggregation mixes) samples are adsorbed in 96 well plates. After blocking the unoccupied sites with albumin, an antibody such as a rabbit anti-SOD antibody is added to the wells. The amount of antibody bound is directly proportional to the amount of SOD-1 adsorbed in the wells. The assay proceeds with the addition of a horseradish peroxidase-conjugated anti-rabbit IgG, that recognizes the anti-SOD-1 antibody, followed by treatment with a color substrate for horseradish peroxidase. The intensity of the color reaction is therefore directly proportional to the amount of SOD-1 and is detected by spectrophotometry.

For the sandwich ELISA technique, a constant amount of unlabelled anti-SOD-1 antibody is adsorbed in the 96-well plate and serves as a capturing reagent. Unoccupied sites are subsequently blocked with albumin. The assay proceeds with the addition of SOD-1 (standard curve and supernatants from control or aggregation mixes) samples followed by an incubation with biotinylated anti-SOD-1 antibody. The amount of biotinylated antibody bound is proportional to the amount of SOD that is bound to the unlabelled (capturing) antibody. After incubating with avidin-horseradish peroxidase, which binds through avidin to the biotin on the biotinylated antibody, wells are treated with a substrate for horseradish peroxidase. The color intensity is directly proportional to the amount of SOD bound to the antibodies and is detected according to standard spectrophotometric methods.

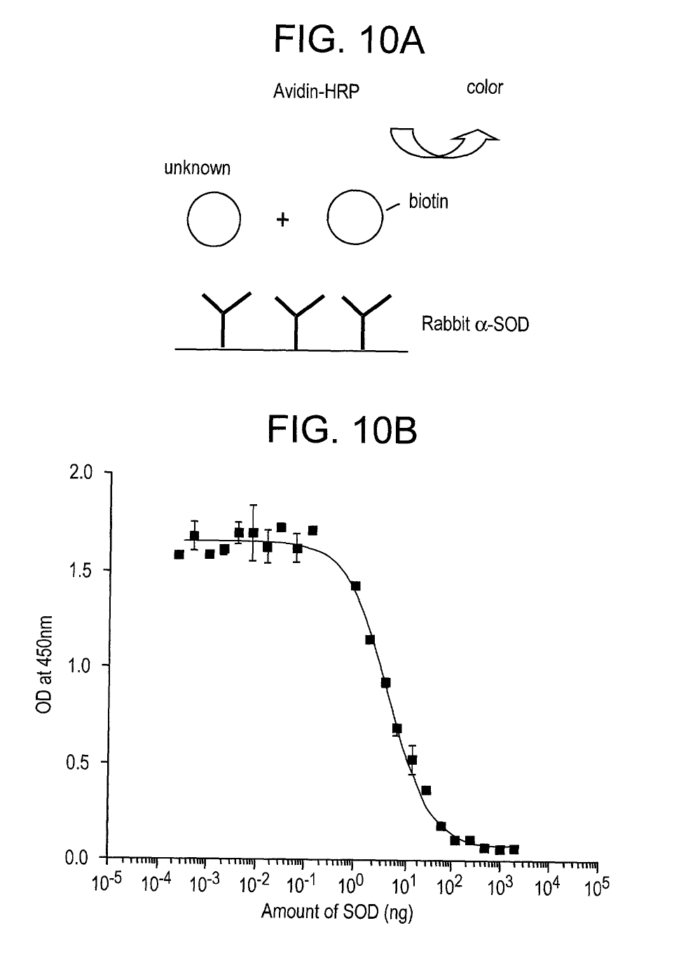

The schematic in FIG. 10A depicts one methodology used to carry out a competitive ELISA. Anti-SOD-1 is adsorbed to wells in a 96 well plate to act as a capture antibody and a mixture of a constant known amount of biotinylated-SOD-1 and an unknown, unlabelled amount of SOD-1 are applied to the well. The amount of biotinylated-SOD-1 bound to the plate is then determined by addition of an avidin-horseradish peroxidase conjugate followed by a substrate for horseradish peroxidase. The amount of color produced is proportional to the amount of biotinylated-SOD-1 that is bound, which, in turn, is proportional to the amount of unlabelled (unknown) amount of SOD-1 present in the competition mixture. Aggregated SOD-1, found in the aggregation mix supernatant, is less able to compete with biotin-SOD-1 for binding to plates as compared to WT, untreated SOD-1since SOD is present in the aggregate and not in the supernatant. Therefore, this assay can be used to measure the relative amount of aggregated SOD-1 present. In a preferred embodiment, this assay can be used to test various agents for their ability to affect SOD-1 aggregation by measuring the ability of MCO-treated SOD-1 to compete with biotin-SOD-1 for binding to plates.

When SOD aggregation is monitored indirectly, for example, by measuring the loss of soluble SOD from a reaction system, a variety of methods well known in the art can be utilized. Exemplary methods for monitoring soluble SOD are described above and include ELISA and optical methods.

Cell Based Aggregation Assays

The above methods describe a variety of in vitro systems that can be used to mimic the misfolding and aggregation of SOD-1 observed in vivo. The present invention also features a cell-based system which can be used to analyze the misfolding and aggregation of SOD-1 and to identify potential SOD-1 aggregation inhibitors in a more physiologically relevant setting. In the in vivo assays, cells are transfected with expression plasmids encoding wild type or mutant forms of SOD-1. The cells used can include any transfectable cell such as mammalian cells (e.g. HEK293A cells, HeLa cells) or insect cells (Sf9 cells). Mammalian cells are preferred.

Transfected cells are then analyzed for SOD-1 aggregate formation. Methods of detecting SOD-1 aggregates in vivo include immunocytochemistry where SOD-1 aggregates show a punctate staining pattern as compared to a uniform type of staining seen with wild type, non-aggregated SOD-1. Antibodies used for immunocytochemistry can include antibodies that recognize SOD-1 itself or antibodies directed against an amino acid tag incorporated into the SOD-1 expression vector (see Example 9). Biochemical assays such as SDS-PAGE, native PAGE, and western blotting can also be used to detect SOD-1 aggregates.

The cell based assay system is a more biologically relevant aggregation system than the in vitro systems. It is a preferred embodiment of this invention that the in vitro based assays described herein be used to identify potentially biologically effective inhibitors of aggregation, and the cell-based assay be used as a secondary screen to determine aggregation inhibition activity in a more biologically relevant system taking into account issues of compound toxicity and cell permeability.

Animal Based Assays

Candidate agents identified in any of the aforementioned assays described above are further screened in standard animal based assays to determine the therapeutic effect of the candidate agent. Exemplary animal based model systems include transgenic mice overexpressing wild type SOD-1 (Epstein et al., Proc. Natl. Acad. Sci., 84:8044, 1987; Gurney et al., Science 264:1772, 1994) or SOD-1 mutations like G93A -SOD-1 (Gurney et al., supra), G37R-SOD-1 (Wong et al., Neuron 14:1105, 1995), G85R-SOD-1 (Brujin et al., Neuron 18:327, 1997) as well as transgenic rats overexpressing SOD-1 mutations such as G93A-SOD-1 and H46R-SOD-1 (Howland et al., Proc. Natl. Acad. Sci., 99:1604, 2002; Nagai et al., J. Neurosci 21:9246, 2001).

Therapeutic Screening—Assays and Agents

The aforementioned SOD-1 aggregation assays are useful for assessing the binding of agents (for example, organic compounds; small molecules; nucleic acid ligands such as DNA, RNA, or mixed nucleotide aptamers; ligands; synthetic chemicals; proteins; agonists; and antagonists) in, for example, chemical libraries and natural product mixtures.

The invention therefore also provides a method of screening agents to identify those that enhance (e.g., an agonist) or block (e.g., an antagonist) aggregation of a SOD-1 polypeptide or that stabilize the native SOD-1 conformation. The method of screening may also involve high-throughput techniques employing standard computerized robotic and microtiter plates as is described below.

In general, the method involves screening any number of agents for therapeutically-active agents by employing the SOD-1 aggregation assays described above. Based on our demonstration that SOD-1 aggregates in vitro, it will be readily understood that an agent which interferes with SOD-1 aggregation in vitro or that reverses the aggregation process or that disrupts SOD-1 aggregates provides an effective therapeutic agent in a mammal (e.g., a human patient).

Accordingly, the methods of the invention simplify the evaluation, identification, and development of active agents such as drugs for the treatment of diseases caused by aberrant SOD-1 aggregation such as ALS.

In general, the chemical screening methods of the invention provide a straightforward means for selecting natural product extracts or agents of interest from a large population which are further evaluated and condensed to a few active and selective materials. Constituents of this pool are then purified and evaluated in the methods of the invention to determine their ability to modulate the aggregation of SOD-1.

Test Extracts and Agents

In general, novel drugs are identified from large libraries of both natural product or synthetic (or semi-synthetic) extracts or chemical libraries according to methods known in the art. The screening methods of the present invention are appropriate and useful for testing agents from a variety of sources for possible activity on SOD-1 aggregation in vitro. The initial screens may be performed using a diverse library of agents, but the method is suitable for a variety of other compounds and compound libraries. Such compound libraries can be combinatorial libraries, natural product libraries, or other small molecule libraries. In addition, compounds from commercial sources can be tested, as well as commercially available analogs of identified inhibitors.

For example, those skilled in the field of drug discovery and development will understand that the precise source of test extracts or compounds is not critical to the screening assays(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semisynthesis or total synthesis) of any number of chemical compounds, including, but not limited to, saccharide-, lipid-, peptide-, and nucleic acid-based compounds, including nucleic-acid ligands such as apatmers. Synthetic compound libraries are commercially available from Nanoscale Combinatorial Synthesis Inc., Mountain View, Calif., ChemDiv Inc., San Diego, Calif., Pharmacopeia Drug Discovery, Princeton, N.J., and ArQule Inc., Medford, Mass. Alternatively, libraries of natural compounds in the form of bacterial, fungal, plant, and animal extracts are commercially available from a number of sources, including Phytera Inc., Worcester, Pa. and Panlabs Inc., Bothell, Wash. In addition, natural and synthetically produced libraries are produced, if desired, according to methods known in the art, e.g., by standard extraction and fractionation methods. Devices for the preparation of combinatorial libraries are also commercially available, for example, Advanced ChemTech, Louisville, Ky. and Argonaut Technologies Inc., San Carlos, Calif. Furthermore, if desired, any library or compound is readily modified using standard chemical, physical, or biochemical methods.

When a crude extract is found to have activity that modulates SOD-1 aggregation in vitro, further fractionation of the positive lead extract is necessary to isolate chemical constituents responsible for the observed effect. Thus, the goal of the extraction, fractionation, and purification process is the careful characterization and identification of a chemical entity within the crude extract having activity that modulates aggregation of SOD-1 (e.g., increases or decreases SOD-1 aggregation). Methods of fractionation and purification of such heterogenous extracts are known in the art. If desired, compounds shown to be useful agents for modulating SOD-1 aggregation in vitro are chemically modified according to methods known in the art to improve their efficacy.

Since many of the compounds in libraries such as combinatorial and natural products libraries, as well as in natural products preparations, are not characterized, the screening methods of this invention provide novel compounds which are active as agonists or antagonists in the particular assays, in addition to identifying known compounds which are active in the screens. Therefore, this invention includes such novel compounds, as well as the use of both novel and known compounds in pharmaceutical compositions and methods of treating disease characterized in aggregation of SOD-1 in vivo such as ALS.

High Throughput Screening Systems

To evaluate the efficacy of an agent (for example, a molecule or an organic compound) in modulating SOD-1 aggregation in vitro any number of high throughput assays may be utilized. The assays are designed to screen large libraries by automating the assay steps and providing compounds from any convenient source to assay, which are typically run in parallel (e.g., in microtiter formats using robotic assays). Thus, by using high throughput assays it is possible to screen several thousand different modulators in a short period of time, for example, 24 hours. In particular, each well of a microtiter plate can be used to run a separate assay against a selected candidate agent that modulates SOD-1 aggregation, or, if concentration or incubation time effects are to be observed, every 5-10 wells can test a single modulator. Thus, a single standard 96-well microtiter plate can assay about 96 modulators. If 1536 well plates are used, then a single plate can easily assay from about 100 to about 1500 different compounds. It is possible to assay many different plates per day; assay screens for up to about 6,000-20,000, and even up to about 100,000-1,000,000 different compounds are possible using computerized robotics.

For example, robotic high-throughput systems for screening of potential modulators of SOD-1 aggregation typically include a robotic armature which transfers fluid from a source to a destination, a controller which controls the robotic armature, a detector, a data storage unit which records SOD-1 aggregate detection, and an assay component such as a microtiter dish comprising a well that includes a SOD-1 aggregation reaction mixture. A number of robotic fluid transfer systems are available, or can easily be made from existing components. For example, commercially-available robotics systems (e.g. TekCel Corporation, Hopkinton, Md.) may be used to set up several parallel simultaneous SOD-1 aggregation assays. Aggregation is detected according to any of the aforementioned detection methods and is optionally processed, e.g., by storing and analyzing the data on a computer. Peripheral equipment and software for storing and analyzing such data are available from Accelrys, San Diego, Calif. and MOE, Chemical Computing Group, Montreal, QC.

For example, to screen for agonists or antagonists of SOD-1 aggregation in vitro, a metal-catalyzing oxidation solution such as ascorbate/Cu²⁺ and SOD-1 are incubated in the wells of a microtiter plate, facilitating the automation or semi-automation of manipulations and full automation of data collection, at 37° C. in the presence and absence of a candidate agent that may be a SOD-1 aggregation agonist or antagonist. The ability of the candidate agent to agonize or antagonize SOD-1 aggregation is reflected in decreased or increased production of SOD-1 aggregates relative to a control sample.

Agents that bind well and increase SOD-1 aggregation are likely good agonists. Agents that bind SOD-1 and inhibit or disrupt aggregation without affecting SOD-1 biological activity are most likely good antagonists of SOD-1 aggregation. Detection of SOD-1 aggregates in solution is accomplished according to any of the above-described detection methods. Preferred detection methods include ANS dye binding or protein staining, RALS, DLS, UV absorption and filter retardation assays

If a candidate antagonist agent is capable of inhibiting or disrupting SOD-1 aggregation, then the level of aggregation detected by any of the assays described above will be reduced in the sample containing the agent compared with the control reaction mixture. Alternatively, increased aggregation relative to a control is indicative of a candidate agonist.

In addition, any candidate agent can be screened using a virtual screening approach. Virtual screening utilizes high-throughput prediction of biological activity based on protein structures or the activity of existing agents in silico. Predicted interactors can then be chemically synthesized and tested in vitro, in vivo, or both. Exemplary virtual screening approaches are described in Stahura et al., (J. Mol. Graph. Model., 20:439, 2002); Schaefer-Prokop and Prokop, (Eur. Respir. J. Suppl., 35:71s, 2002); Toledo-Sherman and Chen, (Curr. Opin. Drug Discov. and Dev., 5:414, 2002); Waszkowycz, (Curr. Opin. Drug Discov. and Dev., 5:407, 2002).

Therapeutics

The methods of the invention provide a simple means for identifying agents capable of either inhibiting or increasing SOD-1 aggregation in vitro. Accordingly, a chemical entity discovered to modulate an increase or decrease in SOD-1 aggregation is useful as either a drug, or as information for structural modification of existing agents that modulate SOD-1 aggregation, for example, by rational drug design.

For therapeutic uses, the agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections which provide continuous, sustained levels of the drug in the patient. Treatment of human patients or other animals will be carried out using a therapeutically effective amount of a SOD-1 aggregation modulating agent in a physiologically-acceptable carrier. In the context of treating ALS a “therapeutically effective amount” or “pharmaceutically effective amount” indicates an amount of a SOD-1 aggregation modulating agent, for example, as disclosed for this invention, which has a therapeutic effect, for example, an agent that inhibits or disrupts SOD-1 aggregation. This generally refers to the inhibition, to some extent, of the normal SOD-1 aggregation behavior causing or contributing to a neurological disorder such as ALS. The dose of the agent which is useful as a treatment is a “therapeutically effective amount.” Thus, as used herein, a therapeutically effective amount means an amount of an agent which produces the desired therapeutic effect as judged by clinical trial results, standard animal models of ALS, or both. This amount can be routinely determined by one skilled in the art. This amount can further depend on the patient's height, weight, sex, age, and renal and liver function or other medical history. For these purposes, a therapeutic effect is one which relieves to some extent one or more of the symptoms of ALS and includes curing the disease.

The compositions containing such agents can be administered for prophylactic or therapeutic treatments, or both. In therapeutic applications, the compositions are administered to a patient already suffering from ALS in an amount sufficient to cure or at least partially arrest the symptoms of the disease. Amounts effective for this use will depend on the severity and course of the disease, previous therapy, the patient's health status and response to the drugs, and the judgment of the treating physician. In prophylactic applications, compositions containing the agents of the invention are administered to a patient susceptible to, or otherwise at risk of, developing ALS as determined from genetic screening. Such an amount is defined to be a “prophylactically effective amount.” In this use, the precise amounts again depend on the patient's state of health, weight, and the like. However, generally, a suitable effective dose will be in the range of 0.1 to 10000 milligrams (mg) per recipient per day, preferably in the range of 10-5000 mg per day. The desired dosage is preferably presented in one, two, three, four, or more subdoses administered at appropriate intervals throughout the day. These subdoses can be administered as unit dosage forms, for example, containing 5 to 1000 mg, preferably 10 to 100 mg of active ingredient per unit dosage form. Preferably, the agents of the invention will be administered in amounts of between about 2.0 mg/kg to 25 mg/kg of patient body weight, between about one to four times per day. Suitable carriers and their formulation are described, for example, in Remington's Pharmaceutical Sciences by E. W. Martin.

The following examples are shown to illustrate, but not to limit the present invention.

### EXAMPLES

SOD-1 Preparation

The following methods were used to prepare SOD-1 for the examples described herein.

Human holo-SOD-1 from erythrocytes

SOD-1 was purchased from Sigma (purified from erythrocytes) and dissolved in the appropriate buffer.

Recombinant human wt holo-SOD-1

The wt SOD-1 construct was generated by cloning the full-length SOD-1 cDNA (ATCC) into the pFLUC (Valentis) mammalian expression vector. Wt SOD-1 cDNA was amplified by PCR to generate ends compatible with the cloning cassette of the vector. Both the PCR product and the expression vector were cut using the compatible enzymes, ligated and transformed in the TOP10 bacterial strain (Invitrogen). Resulting colonies were screened by restriction analysis and confirmed by sequencing.

The wt SOD-1 cDNA was amplified by PCR to generate ends compatible with the pET-3d expression vector (Novagen). Both, PCR products and the pET3d vector were cut with compatible enzymes, ligated and transformed in the BL21(DE3)pLysS bacterial strain (Novagen). All constructs were confirmed by restriction analysis and sequencing.

For bacterial expression of wt SOD-1, bacteria were grown to OD₆₀₀ of 0.6 and induced with 1 mM isopropylthio-β-D-galactoside and grown at 25° C. for 4 hours. CuCl₂ and ZnCl₂ were added to cultures to a final concentration of 50 μM and 100 μM, respectively. Bacteria were centrifuged, resuspended in 20 mM Tris-HCl buffer, pH 8.0, frozen, thawed, DNAse 1 (Sigma) and Complete EDTA-free protease inhibitor cocktail (Roche) were added and the suspension was sonicated for 2×30 seconds. Lysates were centrifuged (13,000×g, 30 minutes) to obtain soluble fractions from which SOD-1 was purified.

Purification of SOD-1 was carried out by diluting soluble bacterial lysates with 5 volumes of 20 mM Tris-HCl buffer, pH 8.0 (buffer A) and ammonium sulfate added to a concentration of 40% saturation at 4° C. with stirring. After 30 minutes, the suspension was centrifuged (23,000×g, 30 minutes) and the supernatant extensively dialyzed against buffer A. The dialyzed supernatant was applied to a column packed with Q-Sepharose HP (Pharmacia, 2.6×12.5 cm) equilibrated in buffer A, and SOD-1 eluted with a linear AB gradient of 0.4% B/min. where buffer B was buffer A containing 1 M NaCl. Fractions containing pure SOD were identified using SDS PADE analysis and were pooled and stored.

Mutant SOD-1

For certain mutants, non-overlapping oligonucleotides (both strands), with one oligonucleotide containing the desired mutation, were synthesized. These were used in conventional PCR reactions where the pFLUC/wt SOD-1 plasmid served as template resulting in full-length linear pFLUC vector containing the mutated SOD-1. The vector was recircularized and the DNA transformed into the TOP10 bacterial strain (Invitrogen). Resulting colonies were screened by restriction analysis and confirmed by sequencing.

Some mutants were also produced using two overlapping oligonucleotides both having the desired mutation. Conventional PCR amplification reactions were carried out and TOP10 bacteria transformed with the PCR products. Resulting colonies were screened by restriction analysis and confirmed by sequencing.

All mutant cDNAs were transferred into pET3d, expressed and purified as described for human wt holo-SOD-1 above.

Apo-SOD-1

Apo-SOD-1 was prepared from holo-SOD-1 as reported (Crow et al., J. Neurochem., 69:1936, 1997). Holo-SOD-1 was first extensively dialyzed against 10 mM sodium acetate buffer containing 1 mM EDTA, pH 3.8 for 24-72 hours, then dialyzed against 10 mM sodium acetate buffer containing 100 mM NaCl, pH 3.8, and finally dialyzed against the desired buffer.

Zinc-deficient SOD-1

Apo-SOD-1 was incubated in the presence of a 1.1-fold molar excess of CuCl₂ in 17 mM sodium acetate buffer, pH 3.8, for 24 hours at 40° C.

### Example 1

### SOD-1 Aggregation Assay Parameters

The effect of pH, copper ions, and ascorbic acid on the induction of SOD-1 aggregation was examined as follows. SOD-1, from human erythrocytes (Sigma-Aldrich Fine Chemicals, St. Louis, Mo., USA), incubated at 37° C. for 38 hours in 10 mM phosphate buffer at pH 6 in the presence of 2 mM ascorbic acid resulted in the formation of aggregates that were detectable by right angle light scattering. Right angle light scattering was measured at room temperature using a Photon Technology International QM-1 fluorescence spectrophotometer. Both the excitation and emission wavelengths were set to 350 nm and a 1 nm bandpass. A cuvette with an excitation and emission path lengths of 2 mm and 10 mm, respectively was used for the measurements.

In addition, right angle light scattering was measured using a DynaPro99 Molecular Sizing Instrument with attached MicroSampler-MSXTC/12 (Protein Solutions, Inc., Charlottesville, Va.). A 12 μl-DynaPro quartz cuvette with a path length of 1.5 mm was used for all experiments. In the measurement, the SOD-1 sample solution was illuminated by a 60 mW, 825 nm solid-state laser, and the intensity of light scattered at a 90° angle was measured directly as the solution photon count rate in units of kcounts/sec. The averaged count rate was automatically calculated after all acquisitions of the measurements.

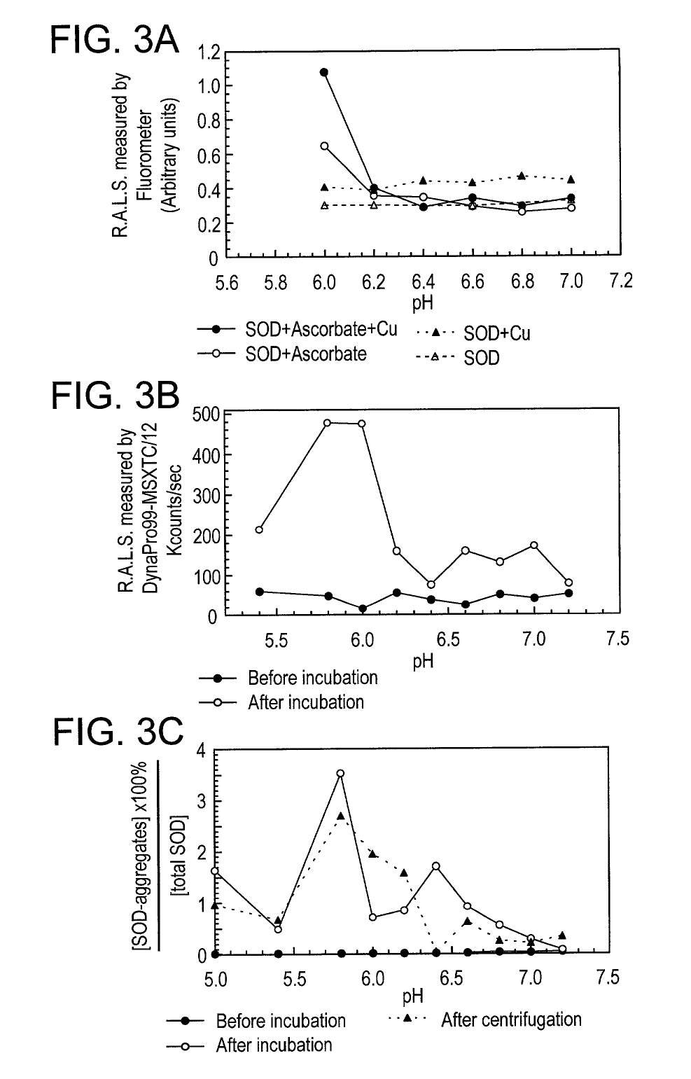

The results of these SOD-1 aggregation studies are shown in FIG. 3A. The relative amount of aggregation induced by ascorbic acid was significantly enhanced by the addition of 25 μM CuCl₂. Moreover, no aggregates of SOD-1 were induced by incubation of SOD-1 in either 10 mM sodium phosphate or 10 mM sodium phosphate containing 25 μM CuCl₂ after 38 hours.

The ascorbate/Cu-induced (MCO) aggregation of SOD-1 was also examined over a broad pH range. SOD-1 from human erythrocytes (Sigma-Aldrich Fine Chemicals, St. Louis, Mo., USA) was incubated at a concentration of 10 μM in four sets of buffers at 37° C. for 38 hours. Each set contained buffers that varied in pH from 5 to 7.2. The first set of buffers were composed of 10 mM sodium phosphate, the second set were composed of 10 mM sodium phosphate and 25 μM CuCl₂, the third set were composed of 10 mM sodium phosphate and 2 mM ascorbic acid, and the fourth set were composed of 10 mM sodium phosphate, 2 mM ascorbic acid, and 25 μM CuCl₂. After incubation, SOD-1 aggregates were detected by right angle light scattering (as described above) and a highly sensitive laser dynamic light scattering method (DLS). DLS measurements were performed on the DynaPro99-MSXTC/12 instrument and data analysis was achieved with DYNAMICS (Version 5.26.38) software supplied with the instrument. The DLS instrument measures fluctuations of the scattered light intensity as a function of time. An autocorrelation function was then used to evaluate the fluctuations in the intensity of the scattered light and calculate the diffusion coefficient of particles in the sample that cause the light scattering. A regularization algorithm was also used to estimate how many different species of scattering particles should be included in the data analysis.

FIGS. 3B and 3C illustrate the results of the right angle laser light scattering and DLS measurements of SOD-1 aggregates with 2 mM ascorbic acid and 25 μM CuCl₂ in 10 mM phosphate buffer. Samples contained 10 μM SOD-1, 2 mM ascorbic acid, and 25 μM CuCl2 in 10 mM sodium phosphate buffer, pH 5.0-7.2. Prior to incubation at 37° C., SOD-1 aggregates were undetectable. After incubation at 37° C. for 40 hours, the right angle light scattering intensity increased in samples in the pH range of 5.8 to 6.0, which indicated the presence of large aggregates in these samples (FIG. 3B). The relative amount of aggregated SOD-1 present was quantified by DLS, using the following procedure. DLS measurements revealed the distribution function of the number and types of particles present in the solution. DLS measurements of SOD-1 samples that were not subjected to ascorbate/Cu treatment and incubation at 37° C. revealed the sole presence of particles of radii around 2.3 nm. These particles appeared to be soluble native SOD-1. DLS measurements of incubated SOD-1 samples revealed the presence of multiple types of particles that ranged in hydrodynamic radii between 10-3000 nm (data not shown). The fraction of SOD-1 molecules that formed aggregates was calculated by dividing the relative abundance (mass %) of particles with radii>10 nm by the relative abundance (mass %) of particles≧2.3 nm. This analysis indicated that aggregated SOD-1 was most abundant in the sample that was incubated for 40 hours at pH 5.8, 37° C. (FIG. 3C); however, aggregated SOD-1 comprised only 3.5% of the total amount of SOD-1 present. The DLS analysis was repeated after centrifuging the incubated samples for 5 min at 13,000×g. Multiple types of particles that ranged in hydrodynamic radii between 10-100 nm were detected. Thus, ascorbate/Cu treatment of SOD-1 induced the formation of both soluble and insoluble aggregates.

### Example 2

### Detection Of SOD-1 Aggregates By Various Biophysical Techniques

A comparison of several biophysical techniques for detecting MCO-induced SOD-1 aggregation was performed as follows. Ten SOD-1 samples were generated. Each sample contained 10 μM SOD-1, 10 mM sodium phosphate, 2 mM ascorbic acid, and 25 μM CuCl₂. Additionally, each sample varied in pH between pH 6 and 7. After a 38 hour incubation period at 37° C., the samples were tested for the presence of aggregates using UV absorption (turbidity) measurements, right angle light scattering, and tryptophan fluorescence measurements.

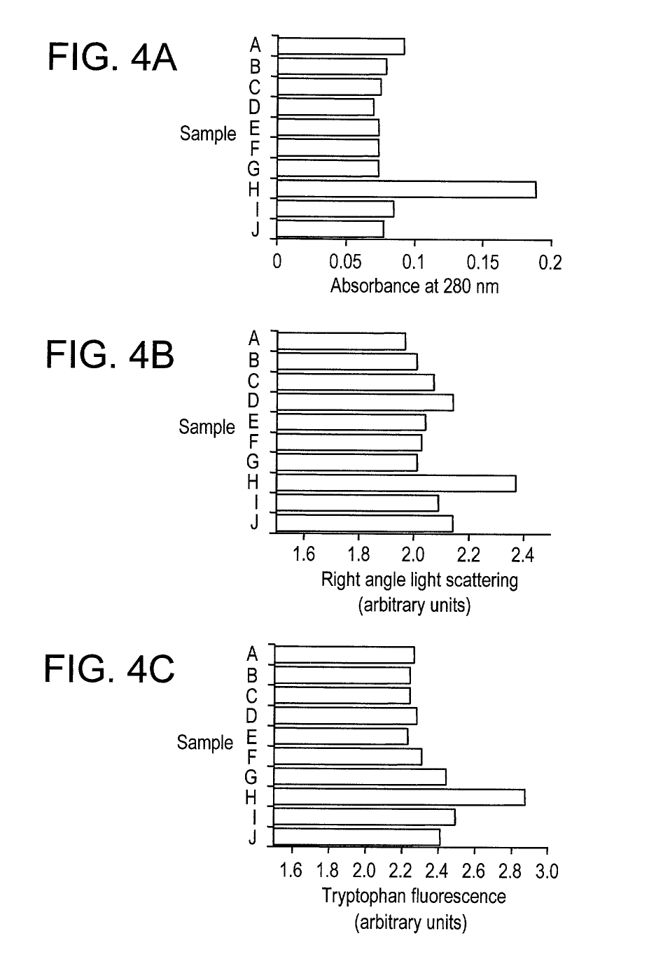

The results of these studies are as follows. To monitor aggregation, the UV absorbance at 280 nm of SOD-1 samples was measured with a Milton Roy Spectronic 3000-diode array UV spectrophotometer. All of the samples contained identical amounts of SOD-1 (10 μM), consequently, any increase in absorbance was likely to be caused by the presence of aggregates that scattered the incident light and increased apparent absorbance. As shown in FIG. 4A, UV absorbance measurements of the incubated samples indicated that sample H possessed greater apparent absorbance than did the other samples, suggesting the presence of aggregates in sample H.

Right angle light scattering measurements, performed as described in Example 1 (above), on the incubated samples also showed the presence of aggregates in sample H (FIG. 4B). These results were similar to those obtained with the UV absorption.

In addition to UV analysis and right angle light scattering measurements, steady-state tryptophan fluorescence of the samples was measured at room temperature using a Photon Technology International QM-1 fluorescence spectrophotometer equipped with excitation intensity correction. For measurements of tryptophan fluorescence, emission spectra from 310 nm to 450 nm were collected (excitation wavelength=280 nm, 0.1 to 1 sec/nm, bandpass=4 nm for excitation and emission). These fluorescence measurements indicated an increase in Trp fluorescence in sample H (FIG. 4C). Taken together with the UV absorption and light scattering measurements, these results showed that SOD-1 aggregation induced an increase in the quantum yield of tryptophan.

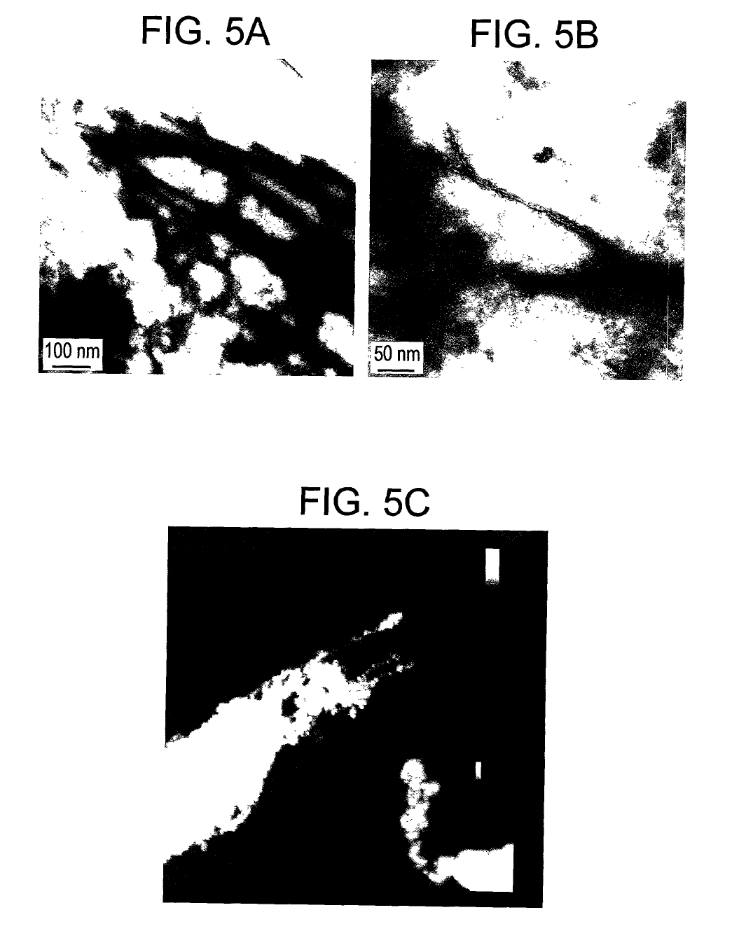

Finally, the ultrastructure of the SOD-1 aggregates was examined by electron microscopy (EM) and atomic force microscopy (AFM). For EM measurements, human wt SOD-1 at a concentration of 30 μM in 10 mM sodium acetate buffer, pH 5.0 was incubated in the presence of 2 mM ascorbate and 25 μM copper at 60° C. for 24 hours. Formvar-coated 220-mesh copper grids (Canemco, Quebec) were floated on 10 μl drops of SOD-1 samples and negative stained with 2% (w/v) uranyl acetate (MecaLab Inc., Quebec). Specimens were examined in a FEI Tecnai 12 transmission electron microscope (80 kV accelerating voltage). These heterogeneous aggregates were composed of amorphous aggregates along with fibrous aggregates that were 40 nm in diameter and several micrometers long (FIGS. 5A and 5B). These fibrous aggregates were thicker than the amyloid fibrils formed by the Alzheimer amyloid peptide, which are 60-90 Å in diameter (Kirschner et al., Proc. Natl. Acad. Sci. 84:6953, 1987). Dye-binding experiments using the thioflavin T were used to determine whether the SOD-1 aggregates possessed amyloid characteristics. A two-fold enhancement of thioflavin T fluorescence was observed with the aggregates produced from zinc deficient SOD-1 (data not shown); however, the fluorescence enhancement seen with amyloid fibrils is usually three orders of magnitude higher (Levine, Protein Sci. 2:404, 1993). Therefore, the SOD-1 aggregates do not appear to be amyloid.

For AFM measurements, oxidation reactions consisted of 10 μM SOD-1, 4 mM ascorbic acid and 0.2 mM CuCl₂ in 10 mM Tris, 10 mM acetate buffer, pH 7.0 whereas control reactions were 10 μM SOD-1 in buffer; reactions were incubated at 37° C. for 48 hours. The image was obtained using a Digital Instruments NanoScope III® atomic force microscope. AFM examination of aggregates formed by oxidation of zinc deficient SOD-1 revealed large amorphous aggregates (<10 μm diameter) that were composed of smaller globular particles (0.5-1.0 μm diameter; FIG. 5C).

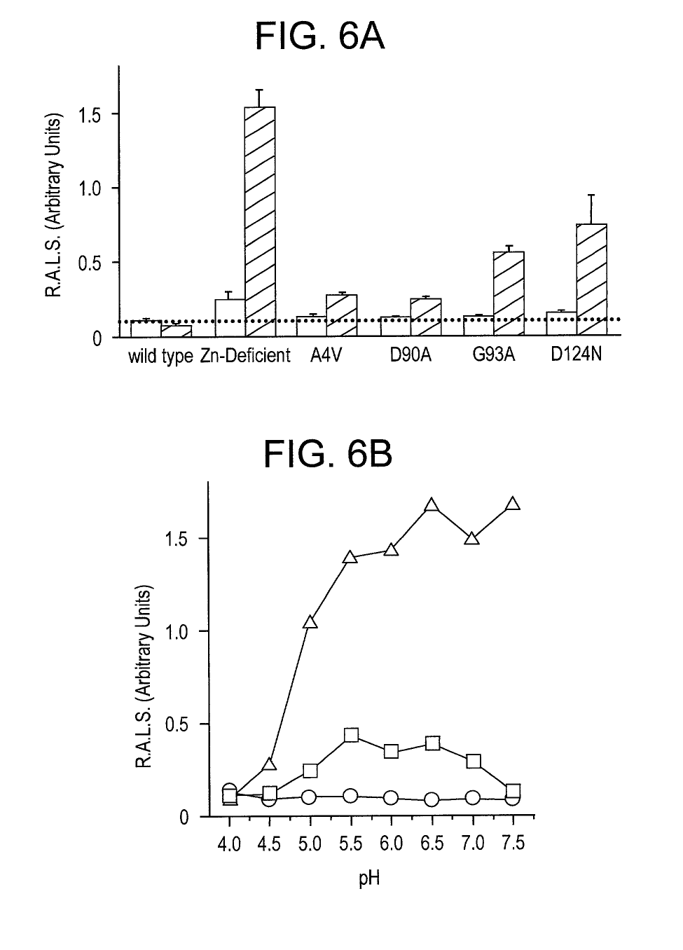

An example of the use of one of these biophysical methods, RALS, to measure and detect SOD-1 aggregation using zinc deficient and mutant forms of SOD-1 is shown in FIG. 6. Oxidation of each of the SOD-1 mutants and zinc deficient SOD-1 induced the formation of visible aggregates that can be detected by RALS. The zinc deficient protein displayed the most robust aggregation reaction, and interestingly D90A, the mutation that causes an autosomal recessive form of FALS, displayed the least amount of aggregate formation. Oxidation of wild type SOD-1 under identical these conditions did not appear to induce the formation of visible aggregates detectable by RALS. With the exception of zinc deficient SOD-1, aggregates were not detected in control samples that lacked oxidants. The small amount of aggregates observed in the control sample of zinc deficient protein suggested that this form of the protein has an intrinsic aggregation tendency. Zinc deficient SOD forms visible aggregates (i.e. >350 nm in diameter) over a large pH range (5.0-7.5) upon oxidation (Δ). Wild type SOD does not form visible aggregates under similar conditions (O). Zinc deficient SOD controls also yielded greater than baseline scattering (□). Aggregates were detected using light scattering measurements made with a Photon Technology International QM-1 fluorescence spectrophotometer. Excitation and emission wavelengths were set to 350 nm (bandpass=4 nm; FIG. 6B). The aggregation reaction displayed distinct pH dependence, with aggregation progressively decreasing at pH values less than 5.5. Similar pH dependence has been observed in the oxidation-induced aggregation of recombinant human relaxin, where the oxidation of a single His residue apparently accounts for the pH-dependence of aggregation (Khossravi et al., Pharm. Res., 17:851, 2000). It should be noted, however, that the optimal pH differs depending on what form of SOD-1 is used as well as what method of detection is employed.

### Example 3

### Detection Of SOD-1 Aggregates By Biochemical Techniques: SDS PAGE, Native PAGE, SEC, Filter Retardation Assay

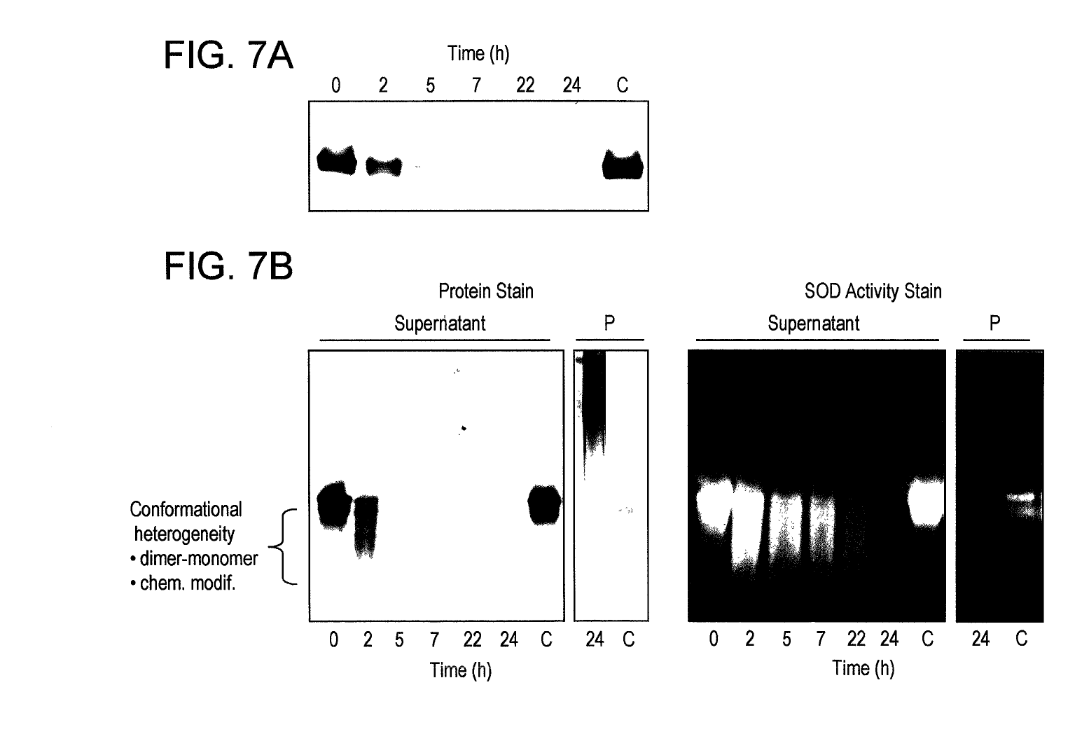

The detection of SOD-1 aggregates was also accomplished using SDS polyacrylamide gel electrophoresis (PAGE), native PAGE, and a filter retardation assay. SDS PAGE and native PAGE were used to measure a time-dependent loss of soluble SOD-1 following treatment with copper and ascorbate (FIGS. 7A and 7B). For SDS PAGE analysis, samples were mixed with an equal volume of 2×SDS PAGE sample buffer, boiled for 5 minutes, and separated on SDS PAGE gels. For native PAGE analysis samples were mixed with an equal volume of 2×native PAGE sample buffer and separated on native PAGE gels lacking SDS. Total protein in gels was stained using coomassie brilliant blue (FIG. 7A) or silver stain (FIG. 7B). SOD activity staining was carried out in a duplicate native PAGE gel by soaking the gel in the presence of nitro blue tetrazolium, riboflavin, and N,N,N′,N′-tetramethylethylenediamine and exposing to fluorescent light as reported (Jia-Rong et al., J. Biochem. Biophys. Methods, 47:233, 2001).

Native PAGE indicated an almost immediate creation of heterogeneously migrating SOD-1 species in the gel. These species may represent chemically modified forms of SOD-1 (i.e. more negatively or less positively charged), monomers of SOD-1, or conformational heterogeneity. As seen in the native PAGE analysis stained for SOD-1 activity (FIG. 7B, right panel), these heterogeneous forms of SOD-1 possessed SOD-1 activity, and that activity loss mirrored the loss of total protein. Analysis of pellets from control samples showed that some SOD-1 was present and was active. This likely represents material that was non-specifically adsorbed to the incubation tube. Analysis of pellets obtained from MCO-treated samples showed SOD-1 present as streaks as well as material that did not enter the gel, consistent with the presence of aggregated material. This aggregated material possessed no SOD-1 activity as would be expected for unfolded and aggregated SOD-1.

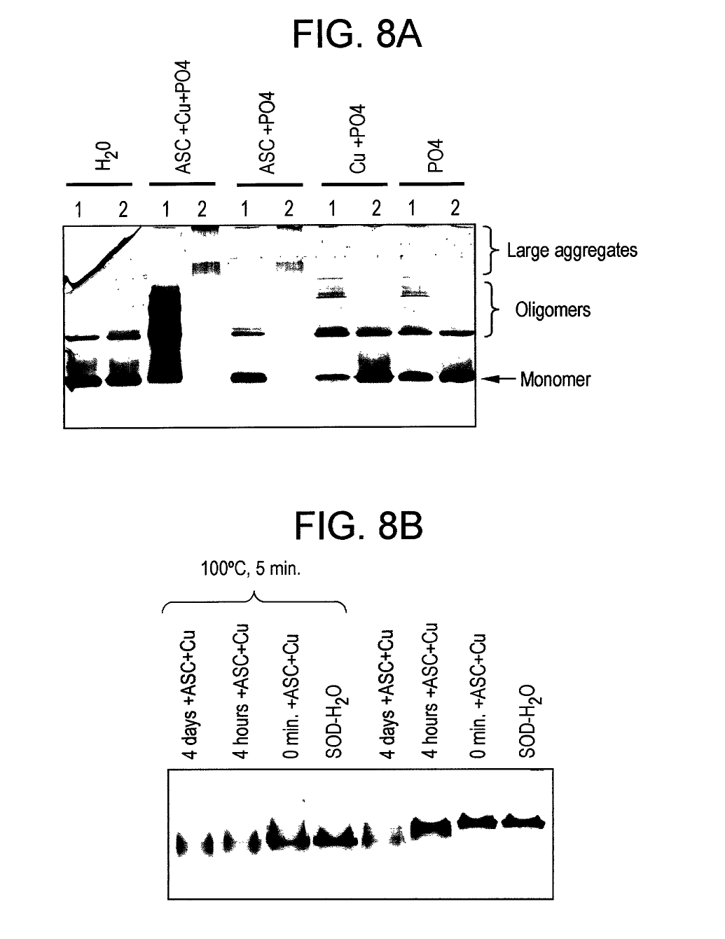

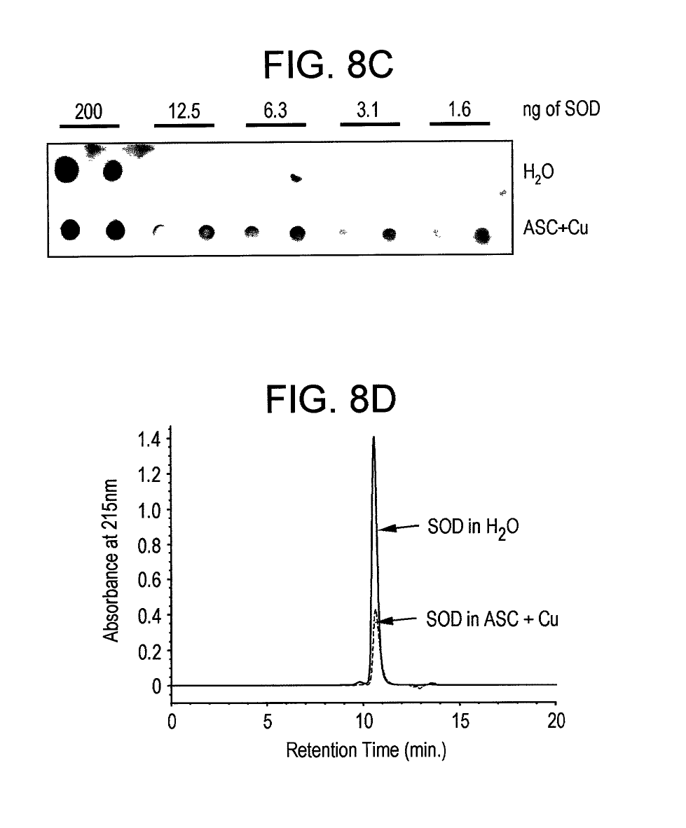

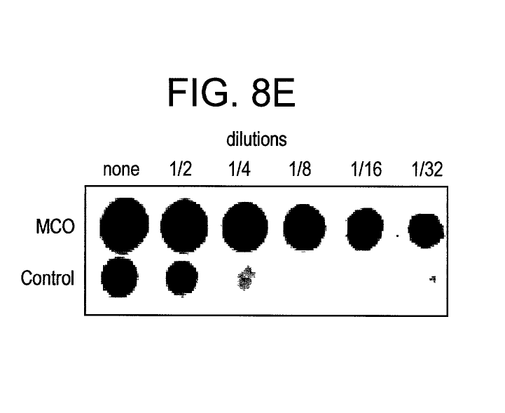

Another example of the use of SDS PAGE and native PAGE electrophoresis to analyze SOD-1 aggregate formation can be seen in FIGS. 8A and 8B. Aggregation samples containing 70 μM SOD-1 in 10 mM sodium phosphate buffer, pH 6.0 were incubated at 37° C. for 6 days prior to analysis in the presence 2 mM ascorbic acid and 25 μM CuCl₂. For SDS PAGE, the incubated samples were mixed with an equal volume of 2×SDS PAGE sample buffer, boiled for 5 minutes, and separated on PAGE gels. As shown in FIG. 8A, SOD-1 dissolved in water alone was found to be predominantly monomeric, but contained a small proportion of dimeric species. Under reducing conditions, dimers were resolved to SOD-1 monomers (data not shown). Following 6 days of incubation in water, the electrophoretic pattern of SOD-1 was substantially identical to that of the freshly prepared sample in water. In contrast, SOD-1 incubated in ascorbate and Cu showed almost immediate formation of oligomers, and after 6 days the sample contained predominantly large SOD-1 aggregates which failed to enter the gel as evidenced by streaking in the well. A similar pattern showing large aggregates was observed when SOD-1 was incubated for six days in an ascorbate buffer (i.e., no exogenous Cu).

Similar SOD-1 aggregation mixtures were also analyzed by native PAGE. Again, aggregation samples contained 70 μM SOD-1 in 10 mM sodium phosphate buffer, pH 6.0, and were incubated at 37° C. for 6 days prior to analysis in the presence 2 mM ascorbic acid and 25 μM CuCl₂. For native page, samples were mixed with an equal volume of native PAGE sample buffer, and either boiled for 5 minutes prior to analysis or not boiled, and separated by native PAGE. As shown in FIG. 8B, SOD-1 dissolved in water migrated as a single species on native PAGE, likely as the native dimer under these conditions. SOD-1 that was incubated in the presence of ascorbate and Cu exhibited a time-dependent increase in migration.

Electrophoretic migration in native PAGE is determined by size, shape and charge of the protein. For analysis by SEC, incubated samples were separated on a Zorbax GF250 size exclusion column (4.6×250 mm) equilibrated with 0.2 M sodium phosphate, pH 6.0, at a flow rate of 1 ml/min. Elution of SOD-1 from the column was monitored at 215 nm. SEC analysis under non-denaturing conditions of SOD-1 in water as compared to soluble SOD-1 remaining in solution following incubation in ascorbate and Cu for 6 days showed that there was no change in size or shape of SOD-1 following incubation (FIG. 8D). The result indicated that the increased migration of SOD-1 observed in native PAGE (FIG. 8B) was due to an increase in the charge to mass ratio, i.e. SOD-1 was becoming more negatively charged (or less positively charged). This result was consistent with the oxidation of specific basic residues within SOD-1, similar to that reported for relaxin by Li et al. (Biochemistry 34:5762, 1995). Furthermore, SEC analysis showed the lack of any small oligomers (e.g., dimers or trimers) as well as an apparent loss of approximately 40% of SOD-1. Native PAGE analysis of identical SOD-1 samples also showed that boiling of the samples enhanced the charge effect, but more significantly, created species that exhibited substantially reduced migration, which are thought to represent SOD-1 oligomers. Thus it appears that boiling of SOD-1 samples introduced artifactual formation of oligomers. Accordingly, methods employing boiling of sample prior to analysis, such as SDS PAGE, are inappropriate for analysis of SOD-1 aggregation.

Another biochemical assay used to measure SOD-1 aggregation was the filter retardation assay for SOD-1 aggregates present in aggregation supernatants as shown in FIG. 8C. Aggregation samples contained 70 μM SOD-1 in 10 mM sodium phosphate buffer, pH 6.0, and were incubated at 37° C. for 18 days prior to analysis in the presence 2 mM ascorbic acid and 25 μM CuCl₂. Aliquots of either supernatants derived from either SOD-1 incubated in water or aggregated SOD-1 were filtered through a 0.2 μm nitrocellulose membrane using a dot blot apparatus (BioRad) under vacuum. The membrane was washed, blocked with tris buffered saline-Tween 20, and SOD-1 trapped on the membrane was detected using anti-SOD-1 antibodies (Stressgen). As shown in FIG. 8C, SOD-1 incubated in water showed retardation on the filtration membrane when 200 ng of SOD-1 was added per well, but no retardation was observed when 12.5 ng or less were filtered. In contrast, up to 1.6 ng of aggregated SOD-1 samples were retained by the nitrocellulose membrane. The differential membrane retention of aggregated SOD-1 compared to SOD-1 in water below a loading of 12.5 ng indicated that large aggregates were present in the aggregation mixture samples and not in SOD-1 dissolved in water. Advantages of such an aggregation assay system are that it can be easily converted to a high throughput methodology and that the method does not rely on boiling of samples that, as shown above, introduce oligomeric forms of SOD-1.

Alternatively, the filter retardation assay can be used to assay aggregated SOD-1 in the insoluble fraction by dissolving the MCO-treated sample pellet in 1% SDS solution. Following incubation as described above, samples were centrifuged (21,000×g, 10 minutes), supernatant removed and 50 μl of a 1% SDS solution in water was added and vortexed thoroughly. Samples were diluted as indicated in 1% SDS. Samples were filtered through a 0.2 μm nitrocellulose membrane (pre-wetted 30 minutes in water) using a dot blot apparatus (Schleicher and Schull). As can be seen in FIG. 8E, SOD-1 was detected in dilutions as large as 1/32 in MCO-treated SOD-1 samples compared to approximately a dilution of ¼ for control samples. Matching of intensities of these dilutions shows that the 1/32 dilution of the MCO-treated sample is approximately the same intensity, and hence contains a similar amount of SOD-1, as the ½ dilution of the control sample. One can estimate therefore that the difference in SOD-1 content between these samples was approximately 15-fold. The large difference in SOD-1 adsorbed to the incubation tubes was due to the aggregation of SOD-1 in the MCO-treated sample. SDS served to solubilize these aggregates to an extent allowing removal from tubes and amounts of aggregated SOD-1 were estimated in this method. Thus, this method represents another screening assay useful for monitoring the accumulation of SOD-1 aggregates, and therefore is also useful for monitoring inhibition of this aggregation process.

Taken together, the above examples demonstrated the effective use of SDS PAGE and native PAGE for the detection of SOD-1 aggregates and further demonstrated that boiling of samples prior to analysis creates artifactual oligomers and should not be used. In addition, the examples also demonstrated the use of SEC and filter retardation assays as effective assays for SOD-1 aggregation when used alone or in combination with any of the other biochemical assays described herein.

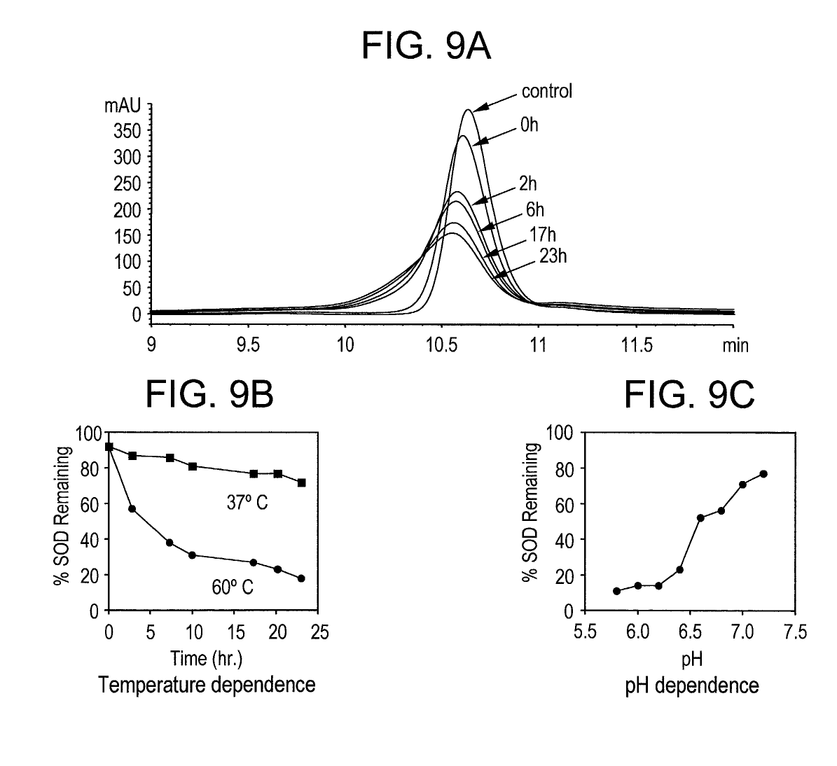

FIG. 9 shows an example of the use of one of these biochemical techniques, SEC, to monitor the loss of soluble SOD-1 as a measurement of SOD-1 aggregation. In order to optimize aggregation conditions, SEC was utilized to monitor the disappearance of soluble SOD-1 under various conditions. SEC was carried out on a Tosohaas TSK 3000 column (4.6×30 cm) using 50 mM sodium phosphate buffer, pH 6.7 as the elution solvent at a flow rate of 0.55 ml/min on an Agilent HP1100 chromatographic system. Detection was by UV absorbance at 215 nm. Loss of soluble SOD-1 was shown to be time dependent (FIG. 9A), temperature dependent (FIG. 9B), and pH dependent (FIG. 9C). This experiment not only demonstrated the critical nature of the time, temperature, and pH when performing SOD-1 aggregation assays, but also demonstrated the effective use of SEC as a measure for SOD-1 aggregation.

### Example 4

### Detection Of SOD-1 Aggregates By Biochemical Techniques: ELISA

Another method used to measure SOD-1 aggregation is the ELISA. The schematic in FIG. 10A describes the methodology to carry out a competitive ELISA. Anti-SOD-1 was adsorbed to wells in a 96 well plate to act as a capture antibody and a mixture of a constant known amount of biotinylated-SOD-1 and a varying amount of an unknown, unlabelled amount of SOD-1 was applied to the well. The amount of biotinylated-SOD-1 bound to the plate was then determined by the addition of an avidin-horseradish peroxidase conjugate followed by a substrate for horseradish peroxidase. The amount of color produced was proportional to the amount of biotinylated-SOD-1 that bound, which, in turn, was proportional to the amount of unlabelled (unknown) amount of SOD-1 present in the competition mixture. When a constant amount of biotin-SOD-1 was mixed with an increasing amount of unlabelled competitor, the amount of biotin-SOD-1 that bound to the plate, and hence the amount of color developed, decreased with an increase in the amount of unlabelled SOD-1 (FIG. 10B).

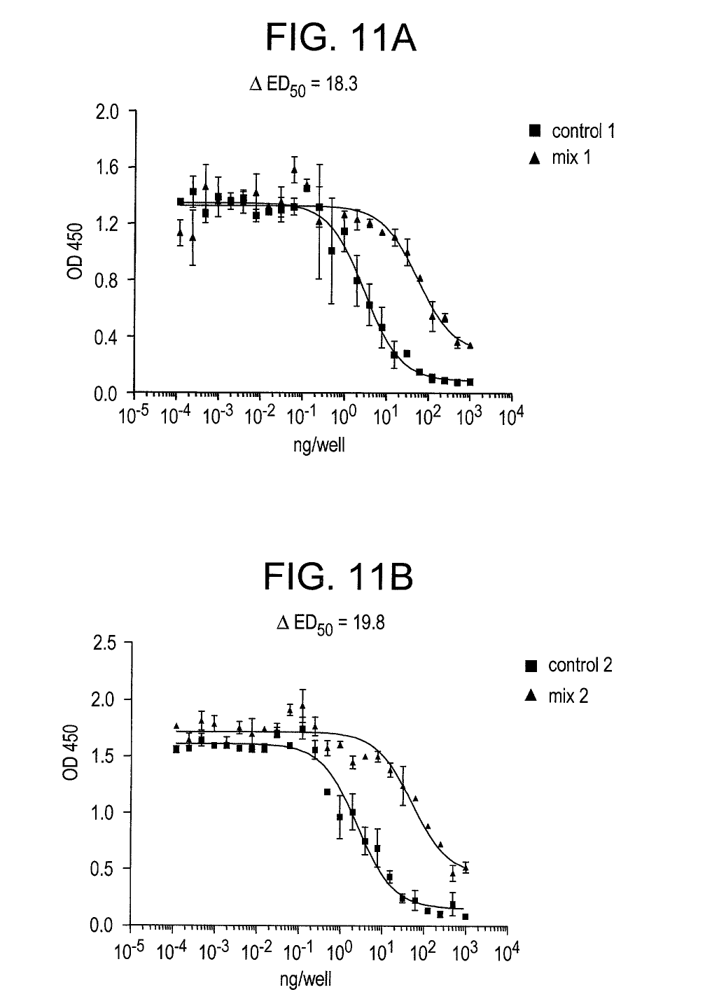

This competitive ELISA technique was used to compare SOD-1 aggregate formation with and without MCO treatment (FIG. 11). Human wt SOD-1 at a concentration of 3 μM in 10 mM Tris-acetate buffer, pH 7.0 was incubated with 5 mM ascorbate and 0.25 μM copper at 37° C. for 24 hours. Control samples were also prepared without the addition of copper and ascorbate. [1] Polyclonal rabbit anti-SOD-1 was purified from sera obtained from rabbits immunized with SOD-1by Protein A chromatography followed by affinity purification on a SOD-1-Sepharose column. [2] Plates were coated with the capture antibody by incubating 50 μl of affinity-purified rabbit anti-SOD-1 at a 1:600 dilution (in PBS) in Immunolon 4HBX 96 well plates (Dynex) overnight at 4° C. Plates were blocked with 1% BSA for 1 hour at room temperature and washed with PBS containing 0.5% Tween 20. [3] To form biotin-SOD, SOD-1 in PBS was added a 5-fold molar excess of EZ-link NHS-LC-biotin (Pierce) dissolved in N,N-dimethylformamide (DMF) to give a final concentration of 12.5% DMF and the reaction mixture incubated at room temperature for 30 min. The reaction mixture was dialyzed against PBS and aliquots stored at −80° C. [4] Competition mixtures were prepared in duplicate containing a constant amount of biotinylated-SOD-1 (2.5 ng) with serial dilutions of unlabelled SOD-1 (based on amounts of SOD-1 input into either control or MCO-treated reactions) and mixtures added to antibody-coated wells in a volume of 50 μl. Plates were incubated at room temperature for 1 hour after which time plates were washed with PBS containing 0.5% Tween 20 and avidin-HRP added to each well followed by incubation for 1 h at room temperature. Plates were then washed as above, TMB peroxidase substrate (KPL) added and color allowed to develop before quenching the reaction with an equal volume of 0.1 M HCl. Plates were read at 450 nm on a plate reader.

Control samples prepared without copper and ascorbate showed a greater ability to compete with biotin-SOD-1 for binding to plates compared to SOD-1 samples subjected to copper and ascorbate treatment (FIG. 11). This is evident since less SOD-1 present in the control mixture (in other words a higher dilution) was capable of reducing color development. Loss of soluble SOD-1 in the MCO-treated samples due to aggregation resulted in the need for the addition of more (or a lower dilution relative to control) SOD-1 to achieve a similar ability to compete with biotin-SOD-1 binding to the plate. Direct comparison of the ED₅₀ values between the curves yielded a relative difference between the amounts of SOD-1 present in control and MCO-treated samples. Under the conditions employed in this example, values in the range of 20-fold differences between ED₅₀ values between control and MCO-treated samples were seen (i.e., a 95% loss of soluble SOD-1 in MCO-treated samples). Depending on aggregation conditions utilized (SOD-1 concentration, copper and ascorbate concentration, time, temperature, pH etc) values ranged from 0- to over 100-fold differences between control and MCO-treated samples.

### Example 5

### MCO-induced Modifications Of SOD-1

An amino acid composition analysis of untreated and MCO-treated SOD-1 was performed to determine which amino acids were affected by MCO treatment. Human wt SOD at a concentration of 30 μM in 10 mM sodium acetate buffer, pH 5.0 was incubated in the presence of 2 mM ascorbate and 25 μM copper at 60° C. for 24 hours and aliquots of the supernatant subjected to amino acid analysis. Control samples were incubated under the same condition in the absence of copper and ascorbate. MCO-treated and control samples were hydrolyzed using 6 M HCl under vacuum at 160° C. for 1 hour, dried under reduced pressure and subjected to amino acid analysis using a Beckman System Gold 6300 high performance analyzer with post-column ninhydrin detection.

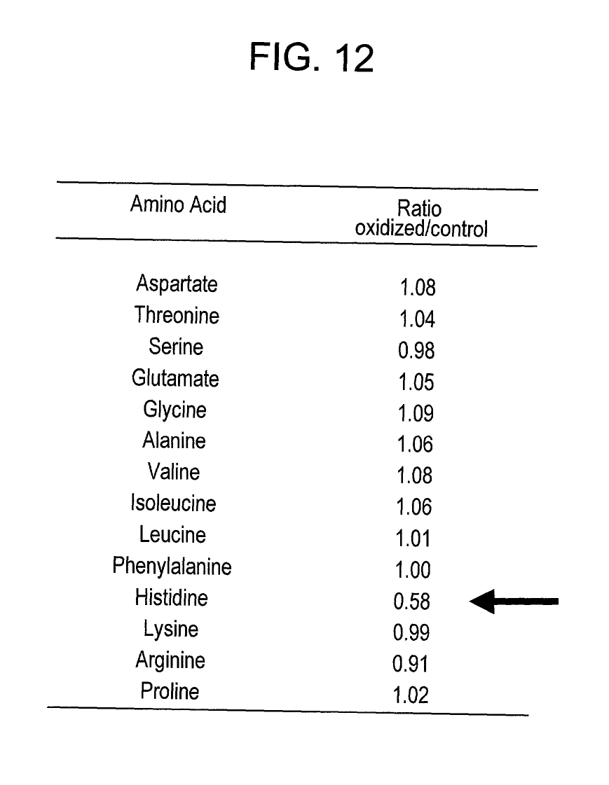

As can be seen in FIG. 12, amino acid composition was identical for MCO-treated and control samples for the majority of amino acids that are not destroyed by the hydrolysis methods utilized. Strikingly, approximately 40% of histidine residues present in control samples were selectively lost from MCO-treated samples suggesting that MCO treatment resulted in damage or changes selectively to specific histidine residues.

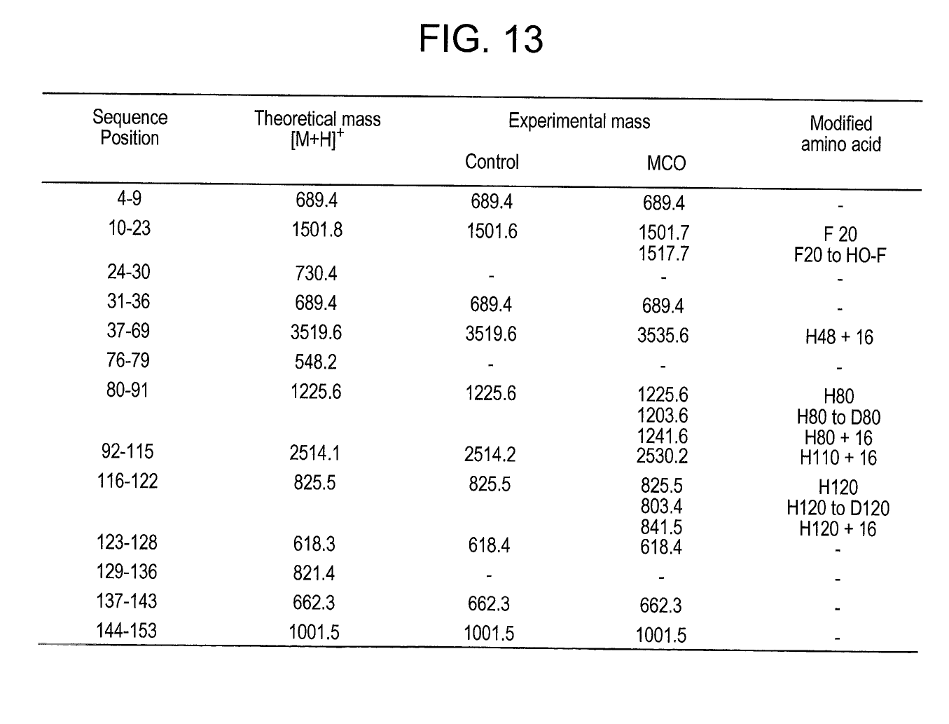

Mass spectrometry analysis was also performed to evaluate changes in amino acid composition of SOD-1 with and without MCO treatment (FIG. 13). Human wt SOD-1 at a concentration of 30 μM in 10 mM sodium acetate buffer, pH 5.0 was incubated in the presence of 2 mM ascorbate and 25 μM copper at 60° C. for 24 hours and aliquots of the supernatant along with control samples not subjected to copper and ascorbate treatment were subjected to tryptic digestion and analysis by capillary LC/MS. For tryptic digestion, samples containing SOD-1 (200 μg, 6 nmol) were dialyzed against a solution containing 1 M Tris/HCl, 6 M guanidine hydrochloride, pH 7.5 for 2 hours and reduced by addition of DTT (300 nmol) and incubated at 50° C. for 1.5 hours. Samples were treated with iodoacetamide (30 μmol) for 1 hour at room temperature, dialyzed against 10 mM acetic acid and lyophilized. Pellets were dissolved in 100 μl of a solution containing 50 mM ammonium bicarbonate, 100 mM urea and 10% (v/v) acetonitrile, trypsin (8 μg, 15 μl) was added, and the solution was incubated at 38° C. for 50 hours. Analysis of ˜1 pmol of tryptic peptides was performed using a Q-TOF Ultima mass spectrometer (Micromass, Manchester, UK) coupled to a capillary HPLC column packed with Jupiter C18 and C4 material. Peptides eluted by acetonitrile were ionized by electrospray and peptide ions were automatically selected and fragmented in a data dependent acquisition mode. Database searching was done with Mascot (Matrix Science) using the oxidation of Met, His and Trp as optional modifications.

FIG. 13 shows the expected peptide fragments following tryptic digestion of SOD-1. The majority of these expected fragments were observed in control samples. Those not observed were either short peptides or very hydrophilic peptides which likely were not retained on the reversed-phase HPLC column. Two of the peptides observed in the control sample (encompassing residues 37-69, expected mass 3519.6 and residues 92-115 with an expected mass of 2514.1) were completely absent in MCO-treated SOD-1, but instead, peptides matching these fragments containing an additional 16 mass units were observed. MS/MS sequencing of these fragments showed that the expected peptide fragments were indeed present but increased in mass by 16 mass units. Sequencing showed that in both these fragments, positions corresponding to histidine residues within the sequences accounted for the increased mass. In this manner it was found that histidine 48 and histidine 110 had been modified. Known oxidative modifications to histidine residues found in proteins include the conversion of histidine to 2-oxohistidine, aspartic acid or asparagine (Berlett and Stadtman, J. Biol. Chem. 272:20313, 1997). Further oxidative modifications were also found for His80 and His120. Oxidized products identified for these residues were conversion to both His containing an additional 16 mass units (likely 2-oxo-histidine) and aspartic acid. Phe20 was also found to be converted to an oxidized form; known oxidative modifications to Phe include conversion to 2,3-dihydroxyphenylalanine, 2-, 3-, or 4-hydroxyphenylalanine (Berlett and Stadtman, supra). It therefore appeared that the loss of histidine residues as determined by amino acid analysis was a result of conversion of these histidine residues to 2-oxo-histidine or aspartic acid. In the case of His48 and His110, the conversion to oxidized product appeared to be quantitative since no peptides with the expected mass were found. In the cases of His80, His120 and Phe20, the conversion of these residues to oxidized products was not quantitative; for His80 and His 120, two different oxidized products were formed.

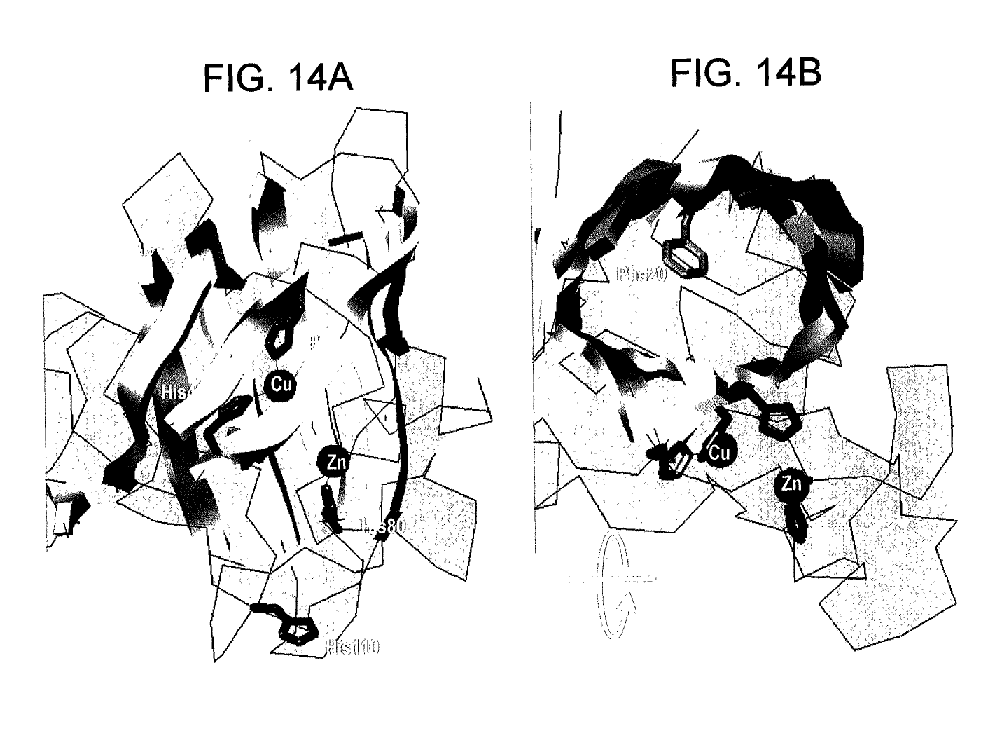

The amino acid analysis data was then used to create a molecular model of human SOD-1 with the oxidized sites as determined by mass spectrometric analysis mapped onto the model structure. As shown in FIG. 14, three of the identified oxidized His residues are located directly in the active site of SOD-1, with two of these normally functioning to coordinate the Cu atom (His48 and His120) and one normally coordinating with the Zn atom (His80). A fourth oxidized His residue is located just outside of the active site (His110). The only other residue found to be oxidized by MCO treatment of SOD-1 was Phe20 (FIG. 13). This Phe residue normally resides in the hydrophobic interior of SOD-1 and is completely solvent inaccessible. Thus oxidation of this Phe would require that the protein unfold, thereby exposing a normally solvent inaccessible residue.

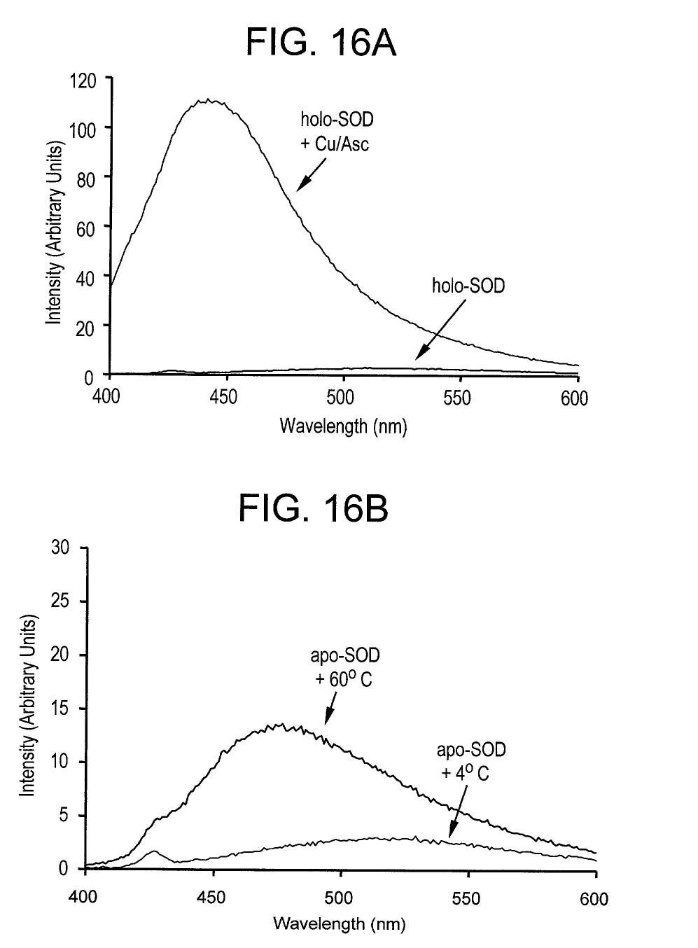

It therefore appeared that the metal-catalyzed oxidation of SOD-1 resulted in the selective oxidation of His residues in or near the active site. It is likely that these modifications occur first and result in the destabilization of SOD-1. Such destabilization was also noted during native PAGE analysis of MCO-treated SOD-1 and indicated the induction of either conformational (structure or oligomerization state i.e. monomer-dimer equilibrium) or chemical heterogeneity in SOD-1. The initial destabilization led to a more global unfolding of the protein allowing the oxidation of normally solvent inaccessible residue Phe20 by the MCO system. The unfolding was also supported by the ability of MCO-treated SOD-1 to bind substantially more ANS relative to control samples (FIG. 16A). The completely unfolded SOD-1 molecules could then go on to assemble into aggregates such as the amyloid-like fibrillar structures seen in FIGS. 5A and 5B.

### Example 6

### Inhibition Of Aggregate Formation By EDTA And Anaerobic Conditions

Zinc-deficient SOD-1 was prepared, MCO-treated, and used to test various compounds and conditions for their effect on SOD-1 aggregation. Zinc-deficient SOD-1 aggregation mixtures were incubated with either 2 mM EDTA, 10 mM mannitol, or 10 mM DMPO as probes for the reactive oxygen species. Anaerobic conditions were achieved by degassing all solutions and oxidizing under vacuum (37° C.) in a vacuum hydrolysis tube (Pierce). Aggregates were detected using light scattering measurements made with a Photon Technology International QM-1 fluorescence spectrophotometer. Excitation and emission wavelengths were set to 350 nm (bandpass=4 nm).

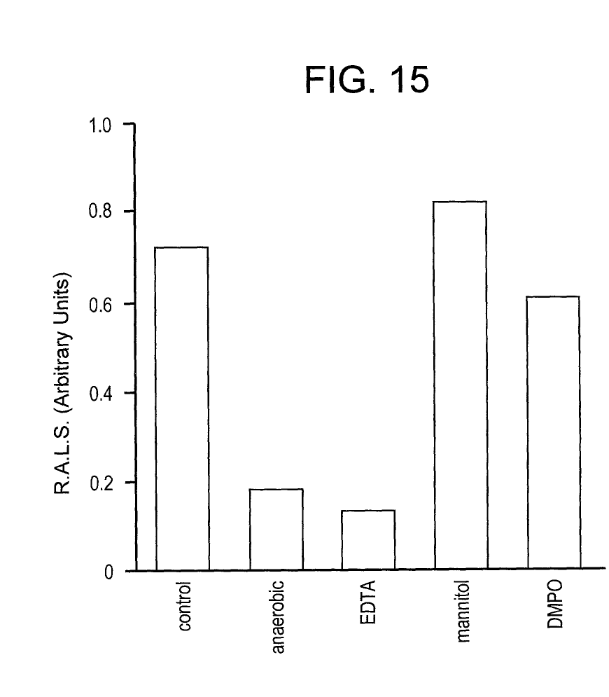

MCO-treated SOD-1 showed clear aggregation as detected by light scattering measurements made with a Photon Technology International QM-1 fluorescence spectrophotometer (FIG. 15). However, performing the oxidation reaction under anaerobic conditions or in the presence of EDTA inhibited aggregation, revealing the absolute requirement of copper and oxygen for aggregation. In contrast, the addition of free radical scavengers, mannitol and DMPO, did not inhibit aggregation. Similar results have been obtained with copper-catalyzed oxidation-induced aggregation of both human relaxin (Li et al., supra) and hamster prion protein (Requena et al., Proc. Natl. Acad. Sci. 98:7′70, 2001). The insensitivity to free radical scavengers and the pH dependence of the oxidation-induced aggregation are consistent with the site-specific metal-catalyzed oxidation mechanism, in which there is a requirement for a metal ion binding site that is in close spatial proximity to the modification sites (Berlett and Stadtman, supra). In this type of oxidation reaction, very few residues are modified.

### Example 7

### Characterization Of The Folded State Of SOD-1 Under Various Conditions

ANS dye binding assays were carried out to characterize the folded state of SOD-1 under various conditions. ANS (1-anilinonaphthalene-8-sulfonic acid) is a dye that fluoresces weakly under aqueous conditions but exhibits both a blue shift as well as greatly increased fluorescence intensity in the presence of hydrophobic surfaces; it has been used extensively to probe for the exposure of hydrophobic surfaces on proteins as an indicator of protein unfolding. As shown in FIG. 16A, in the presence of wt holo-SOD-1, ANS exhibited essentially no fluorescence as is expected for a properly folded protein with hydrophobic groups sequestered in the interior of the protein. Treatment of holo-SOD-1 with copper and ascorbate resulted in a blue shift and increased fluorescence intensity indicating the presence of exposed hydrophobic groups and hence unfolding of SOD-1. Similarly, apo-SOD-1 exhibited little interaction with ANS when incubated at a low temperature but did exhibit increased fluorescence and the characteristic blue shift when mixed with apo-SOD-1 incubated at 60° C. (FIG. 16B). These findings indicated that incubation of apo-SOD-1 at 60° C. resulted in the unfolding of SOD-1. Thus, both metal-catalyzed oxidation of holo-SOD-1 and incubation of apo-SOD-1 at 60° C. resulted in the destabilization and unfolding of SOD-1. ANS binding is a very sensitive probe for unfolding and aggregation and can be readily used to monitor the aggregation process and hence also as a screen for monitoring the inhibition of aggregation. ANS is particularly useful as a screen for proteins that function to stabilize the native, folded state of SOD-1.

### Example 8

### Native State Stabilization Assays

The native state stabilization assay is a complementary assay to the MCO-mediated aggregation assay. This assay is not based on direct chemical modification of SOD-1 to cause aggregation, but rather, is based on the ability of destabilized SOD-1 to unfold and aggregate. Destabilization of SOD-1 can be carried out in any of a number of ways, including: 1) using apo-SOD-1, or Zn-deficient SOD-1 as these are less stable than holo-SOD-1 based on differential scanning calorimetric measurements (Rodriguez et al., supra); 2) by applying a form of stress that does not lead to chemical modifications—examples of such non-modifying methods include thermal or chemical (such as in the presence or urea or guanidine hydrochloride) conditions.

Any compound which binds the native form of SOD-1 would be expected to stabilize that native form by virtue of binding (see FIG. 2C). Stabilization of the native state of SOD-1 by binding of a small molecule is expected to prevent or reduce unfolding and aggregation both in vitro and in vivo. Small molecules that bind to and stabilize native SOD-1 can be identified either through direct binding studies to find interacting molecules, or indirectly by screening for agents that prevent or reduce unfolding and aggregation due to the application of any form of denaturation stress such as heating or chemical denaturants.

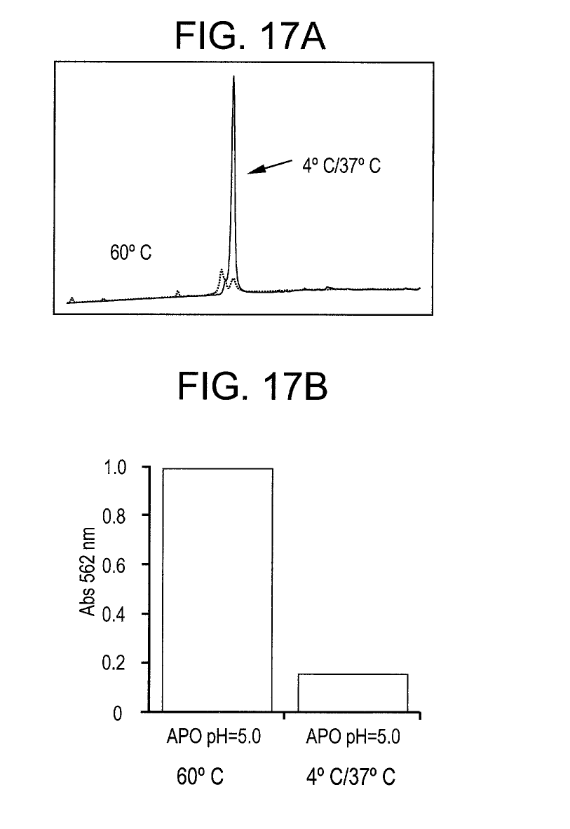

For FIG. 17, apo-SOD-1 (100 μl) prepared as described above at a concentration of 30 μM in 10 mM sodium acetate buffer, pH 5.0, was incubated for 24 hours at either 4°, 37°, or 60° C. in sealed polypropylene tubes. As can be seen in FIG. 17A, samples incubated at either 4° or 37° C. contain the same amount of SOD-1 present in the supernatants as measured by RP-HPLC analysis. RP-HPLC was carried out on a Zorbax SB300 C8 column (3.0×10 cm, 3.5 μm particle size) using a linear 4% B per min AB gradient at a flow rate of 1.0 ml/min, where A was 0.1% aqueous TFA and B was 0.1% TFA in acetonitrile. Detection was based on UV absorbance at 215 nm. In contrast, the sample incubated at 60° C. contained essentially no soluble SOD-1 in the supernatant as measured by the same method of analysis. Since the incubation temperature of 60° C. is close to the melting temperature of apo-SOD-1 (Rodriguez et al., supra), incubation at 60° C. resulted in the thermal-induced unfolding and aggregation of SOD-1. Samples incubated at either 4° or 37° C. on the other hand were not thermally unfolded and hence remained in solution.

Thermal-induced aggregation of apo-SOD-1 can also be monitored by measurement of the amount of aggregated material directly using a protein staining technique (FIG. 17B). Samples were prepared exactly as for FIG. 17A, supernatants were removed, tubes were washed 4 times with 10 mM sodium acetate buffer, pH 5.0, and washes were discarded. To the tubes was added 100 μl of micro BCA protein determination reagent (Pierce), the tubes sealed and incubated at 60° C. for up to 1 hour to allow for color development. The amount of protein present was quantitated by measuring absorbance at 562 nm.

Whereas little staining was seen in samples incubated at either 4° or 37° C., substantially greater staining was seen in the sample incubated at 60° C. indicating that more SOD-1 had aggregated and been deposited on the sides of the tubes. Thus, either the loss of soluble SOD-1 can be monitored to indirectly measure aggregation, or the accumulation of aggregated SOD-1 can be monitored directly to measure the thermal-induced unfolding and aggregation of SOD-1. Carrying out these assays in the presence of small molecule agents allows for the identification of agents that are capable of binding to and stabilizing the native structure of SOD-1 as the loss of soluble SOD-1 to aggregation will be decreased or the amount of aggregate formed will be decreased.

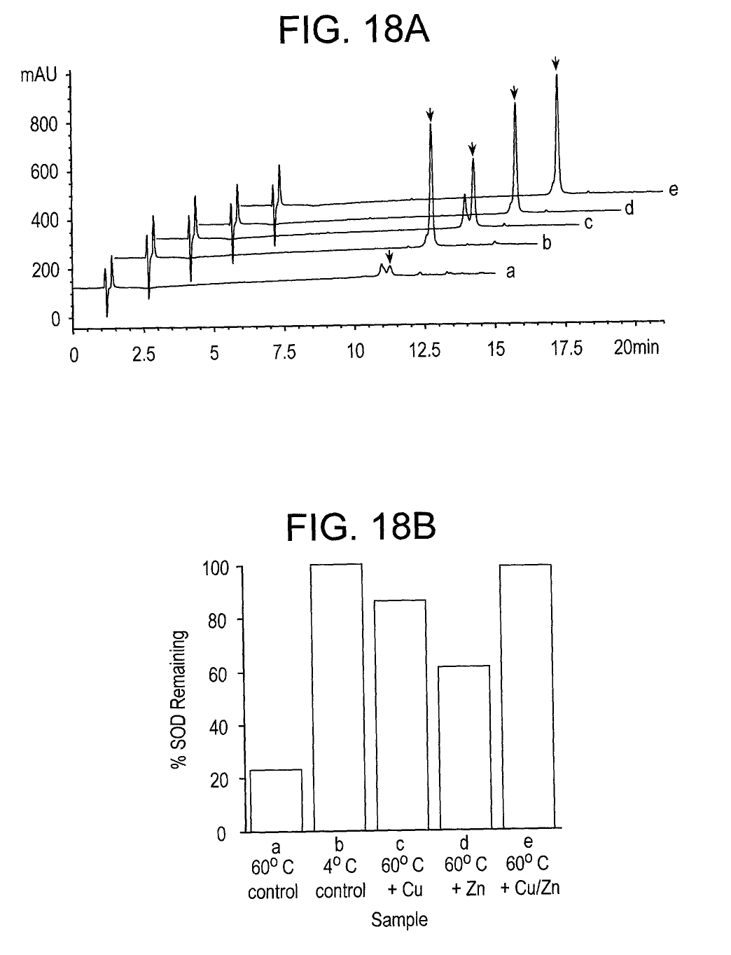

An example of the use of the native state stabilization assay to test agents for inhibition of aggregation can be found in FIG. 18. Apo-SOD-1 samples were incubated under various conditions and analyzed by RP-HPLC to quantitate amounts of soluble SOD-1 remaining. Apo-SOD-1 incubated at 4° C. (negative aggregation control; chromatogram b) showed substantially more SOD-1 present in the supernatant compared to the sample incubated at 60° C. (positive aggregation control; chromatogram a). Apo-SOD-1 incubated at 60° C. in the presence of exogenous zinc or copper or a mixture of copper and zinc (chromatograms c, d and e) were found to possess significantly more soluble SOD-1 than when apo-SOD-1 was incubated at 60° C. in the absence of any exogenous metal (chromatogram a) indicating that the metals were highly effective in preventing aggregation of apo-SOD-1. The addition of copper was found to produce a second species with a slightly lower retention time than SOD-1 which may represent monomeric SOD-1. Quantitation of aggregation inhibition shown in FIG. 18B indicated that the most effective treatment in preventing aggregation was the addition of both copper and zinc which showed essentially 100% effectiveness. This result confirms that both copper and zinc would bind to SOD-1 and occupy their native metal binding sites converting apo-SOD-1 to holo SOD-1, thereby increasing thermal stability. Whereas apo-SOD-1 has a Tm of approximately 60° C., holo-SOD-1 has a Tm of approximately 80° C. (Rodriguez et al., supra). The addition of either metal alone was less effective than both metals in combination in stabilizing apo-SOD-1 and preventing loss of SOD-1 due to aggregation. This example shows that a similar methodology can be used to screen non-natural ligands for SOD-1 to identify agents that bind to, and stabilize the native state of SOD-1. Agents can either bind to the metal-binding site or elsewhere on the surface of the molecule to produce the same effect. It is likely that different reagents used in the assay (apo-, Zn-deficient or holo-SOD-1) will identify different ligands since the metal-binding sites of these reagents will be occupied to different degrees and allow access to different agents.

### Example 9

### Cell Based Aggregation Assay

SOD-1 aggregation assays can also be performed in an in vivo system using a cell based aggregation assay. HEK293A cells in D-MEM at a density of 25,000 cells/well (8-multiwell chamber) were transfected with HA-tagged human wt SOD-1 or HA-tagged human mutant SOD-1 cDNA contained in the pFLUC plasmid (Valentis) by mixing 1 μg of plasmid DNA with 3 μl of Fugene 6 (Roche) in a final volume of 100 μl. The DNA-Fugene 6 mixture was incubated for 25 min. at room temperature prior to addition of 10 μl of the mixture to the wells containing HEK293A cells after which time cells were incubated (37° C., 5% CO2) for 48 hours prior to analysis.

Cells were fixed using formalin (3.7% in PBS) for 20 minutes, washed with PBS and slides blocked with 5% normal serum in PBS for 20 min. Rat monoclonal anti-HA (Boehringer) diluted to 1 μg/ml in PBS containing 0.02% Triton X-100 (wash solution) was added following removal of blocking reagent. Slides were incubated for 1 h, washed, a 1:500 dilution wash solution of Cy2-conjugated goat anti-rat IgG (1:1000) added and slides incubated for 45 min. in the dark. Slides were washed and mounted in Prolong (Molecular Probes). Samples were examined in an inverted Zeiss Axiovert 200 microscope, using a Tex Red filter and 40×, 63×, or 100×apo-chromat Zeiss objectives and a 10×eyepiece. Images were captured using an AxioVision 3.0 program.

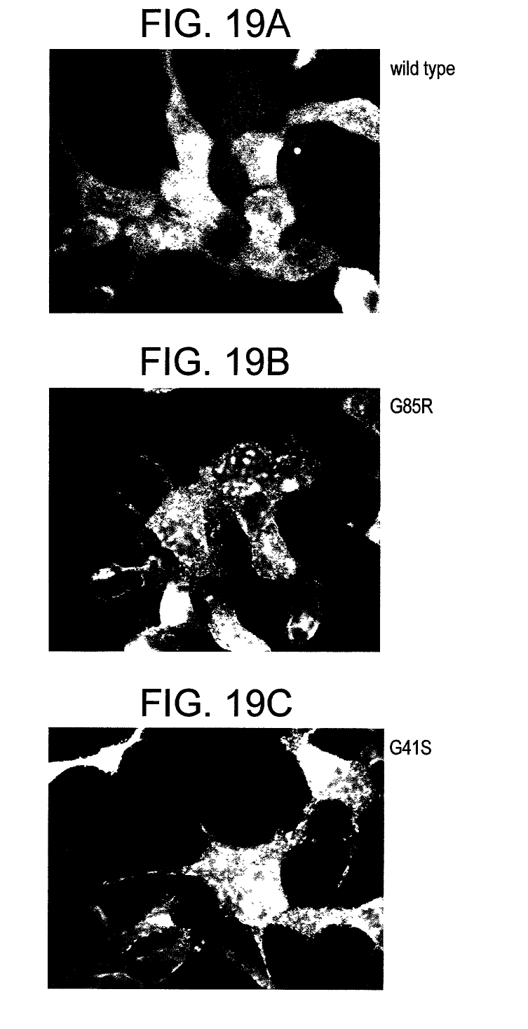

Overexpression of human wt SOD-1 in HEK293A cells resulted in a uniform staining for HA-tagged SOD-1 (FIG. 19A). Overexpression of two different mutants in these cells however resulted in a punctate staining pattern for HA-tagged SOD-1 indicative of formation of SOD-1 aggregates (FIGS. 19B and 19C). Such aggregates have been previously reported following transfection of non-neuronal eukaryotic cells (Koide et al., Neurosci. Lett. 257:29, 1998; Johnston et al., supra) or cultured motor neurons (Durham et al., supra) with mutant forms of SOD-1.

For biochemical characterization of SOD-1 aggregation in vivo, transfections were performed as described above. For cells treated with the proteasome inhibitor ALLN, 1.5 μl of a 20 μg/μl stock solution of ALLN (Calbiochem) was added to wells 36 hours following transfection (final concentration of ALLN was 10 μg/ml) and cells were analyzed 12 hours later.

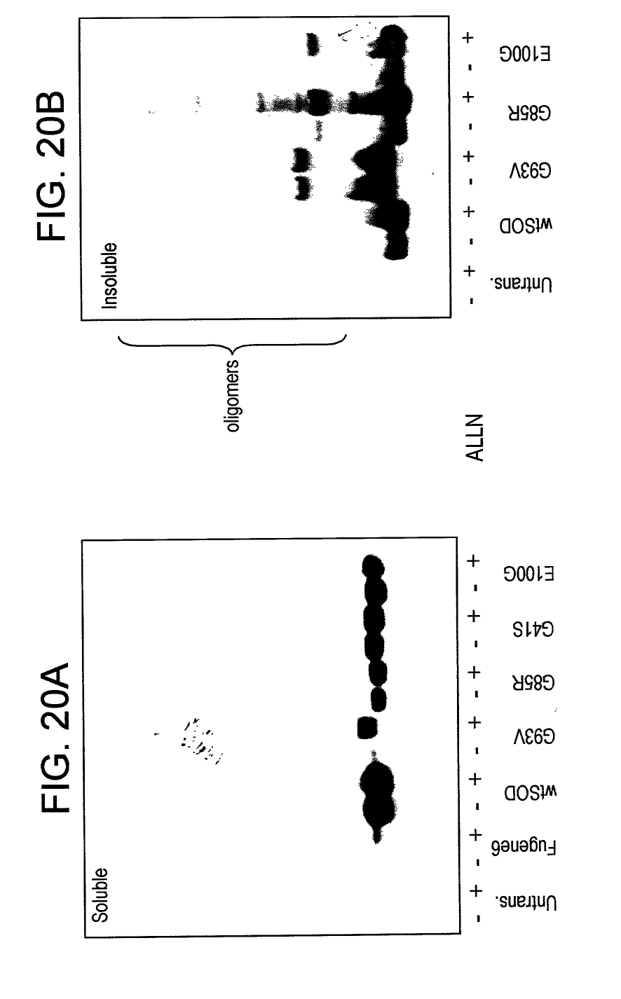

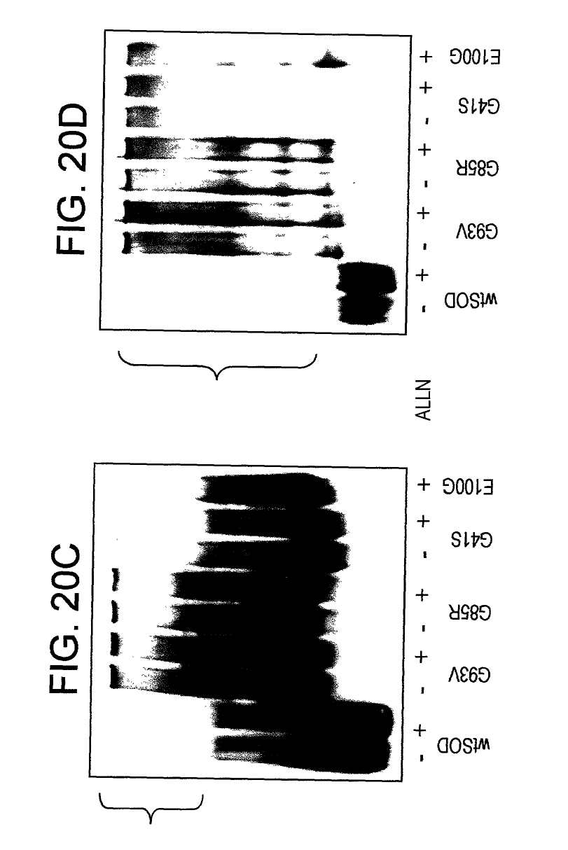

Cells were transfected with either wt or mutant SOD-1 and the presence of SOD-1 in aqueous soluble (no detergents) or insoluble cell fractions was determined. Cells were washed with PBS, 80 μl of PBS containing complete protease inhibitor cocktail (Roche) added, and cells scraped from the surface of the plates with a cell scraper. Cells were sonicated for 8 seconds and centrifuged, the supernatant removed (soluble fraction) and the pellet further sonicated for 8 seconds in 40 μl of PBS containing protease inhibitor (insoluble fraction). Both soluble and insoluble fractions were mixed with equal volumes of appropriate PAGE sample buffer and analyzed by SDS PAGE/Western blot analysis under reducing conditions (FIGS. 20A and 20B) or native PAGE/Western blot analysis (FIGS. 20C and 20D). The primary antibody used for detection of SOD on Western blots was rabbit anti-SOD (Stressgen).

HEK293A cells transfected with mutant SOD-1 contained both monomeric as well as oligomeric forms of SOD-1 in their insoluble fractions when analyzed by SDS PAGE (FIG. 20B). This is in contrast to wt SOD-1-transfected cells where only one band with the expected molecular weight was seen. The oligomeric forms of mutant SOD-1 do not appear to be a result of disulfide-mediated crosslinking as analysis was carried out under reducing conditions. This suggested that the oligomers formed a relatively strong self-association. Treatment of the cells with the proteasome inhibitor ALLN following transfection increased the amount of oligomeric SOD-1 formed with some mutants. The presence of the proteasome inhibitor has previously been shown to increase the steady-state levels of SOD-1 mutants expressed in HEK293A cells, likely as a result of decreased degradation of mutants due to decreased stability or decreased folding efficiency (Johnston et al., supra). Interestingly, no oligomeric forms of SOD-1 were seen in the soluble fraction from these cells.

Analysis of these same fractions under native conditions i.e. in the absence of SDS, showed that oligomeric forms of SOD-1, some too large to enter the gel, were present in both soluble and insoluble fractions of mutant-transfected HEK293A cells (FIGS. 20C and 20D). Transfection with wt SOD-1 did not result in the formation of these oligomers. The presence of oligomers in the soluble fraction of native PAGE gels suggests that boiling the sample in SDS prior to SDS PAGE analysis resulted in the dissolution of these oligomers since none were seen in the soluble fractions analyzed by SDS PAGE.

Mutant SOD-1 oligomers and aggregates can therefore be readily formed by transfection of HEK293A cells with mutant SOD-1s. Aggregates can then be detected using either immunocytochemical detection or a biochemical assay employing PAGE detection of oligomers or higher aggregates. The ability to readily create and monitor SOD-1 oligomerization in cells offers an opportunity to utilize such a system to identify aggregation inhibitors. Such a cell-based assay system is a more biologically relevant aggregation system compared to the in vitro aggregation assays described above since this system takes into account issues of compound toxicity and cell permeability. It is envisioned that the in vitro (whole molecule) assays serve as a high throughput method to identify potentially biologically effective aggregation inhibitors, whereas the cell-based assay serves as a secondary screen to determine aggregation-inhibition activity in a more biologically-relevant system. The next step would be to test agents showing activity in the cell-based assay system in vivo using an animal-based assay system.

### Example 10

### Methods For Identifying An Aggregation Inhibitor Using MCO Based In Vitro Assays

SOD-1 is prepared and purified as described in the examples above. Agents to be tested are added to the SOD-1 sample. Test agents are generally added in a carrier vehicle (e.g., DMSO, ethanol, DMF). Ascorbic acid and CuCl₂ are added to the sample. Concentrations of ascorbic acid and CuCl₂ can vary but are generally 2 mM and 0.25 to 25 μM, respectively. Samples are incubated at 37° C. for 24 hours and aggregated SOD-1 is measured by any of the methods of detection described herein. Any agent that reduces or inhibits the formation of SOD-1 aggregates is considered a potential SOD-1 aggregation inhibitor.

As a positive control for the assay, the SOD-1 sample is prepared as described above, however, only the carrier vehicle is added to the sample instead of carrier vehicle plus the test agent. This sample will be used as a positive control for the reaction conditions and should show MCO-induced SOD-1 aggregation.

As a negative control for the assay, the SOD-1 sample is prepared as above in the absence of any test agent and in the absence of ascorbic acid and CuCl₂. Without ascorbic acid and CuCl₂, SOD-1 aggregates will not form as is demonstrated in FIG. 3A.

Potential inhibitory agents identified in the above assay are then tested in additional assays using various methods of detection to confirm the inhibitory nature of the agent. In addition, agents that inhibit SOD-1 aggregation in vitro can also be tested in the cell based assays described in Example 9. This in vivo assay is used to test the agent in a more biologically relevant setting.

### Example 11

### Methods For Identifying An Aggregation Inhibitor Using Native State Stabilization Assays

Native state stabilization assays are also useful for screening for agents that inhibit aggregation. The native state stabilization assay is not based on direct chemical modification of SOD-1 to cause aggregation, as is seen with the MCO-induced aggregation, but instead is based on the ability of destabilized SOD-1 to unfold and aggregate. Potential aggregation inhibitors in the native state stabilization assay can function either to inhibit the aggregation caused by destabilization, or they function by binding to the destabilized or native state of SOD-1 and preventing it from aggregating.

In this assay apo-SOD-1, zinc deficient SOD-1 or any other form of SOD-1 known to unfold and aggregate under conditions described in Example 8, is prepared and purified as described above. Agents to be tested are added to the SOD-1 sample. Test agents are generally added in a carrier vehicle (e.g., DMSO, ethanol, DMF). The sample is then incubated under conditions that induce aggregation (e.g., for apo-SOD-1 the sample is incubated at 60° C., pH 5 for 24 hours). SOD-1 aggregation is then measured by any of the methods of detection described herein. Any agent that reduces or inhibits the formation of SOD-1 aggregates is considered a potential SOD-1 aggregation inhibitor.

As a positive control, the SOD-1 sample is prepared and incubated exactly as described above, however, only the carrier vehicle is added to the sample instead of carrier vehicle plus the test agent. This sample is used as a positive control for the reaction conditions and will show maximal SOD-1 aggregation.

As a negative control, the SOD-1 sample is prepared as described above, but it is not incubated under conditions that induce aggregation. Instead the sample is incubated under conditions that are known not to induce aggregation (e.g. for apo-SOD-1 the sample is incubated at 4° C., pH 5 for 24 hours; see FIG. 17). This sample should not demonstrate any SOD-1 aggregation and serves as a control for any non-specific SOD-1 aggregation.

Potential inhibitory agents identified in the above assay are then tested in additional assays using various methods of detection to confirm the inhibitory nature of the agent. In addition, agents that inhibit SOD-1 aggregation in vitro can also be tested in the cell based assays described in Example 9. This in vivo assay is used to test the agent in a more biologically relevant setting.

In addition, stabilizing agents are also by identified by screening agents that bind to SOD-1. In order to test for agents that bind to the native form of SOD-1 and prevent aggregation, test agents are first screened for the ability to bind to SOD-1 using any art-known binding assays. Some examples include Biacore measurements in which the potential ligand is immobilized and a SOD solution passed over to measure binding; binding of radio-, fluorescently- or biotin-labeled compounds to immobilized SOD-1 or immobilizing ligands and looking for binding of labeled SOD-1 (label could be biotin, fluorescent tag, biotin etc) or alternatively detecting SOD-1 immunologically in an ELISA type assay. Any SOD-1 binding agent identified is predicted to stabilize the native state of SOD-1 and is then tested in the aggregation inhibitor assays described above.

### Other Embodiments

All publications and patents mentioned in this specification are herein incorporated by reference to the same extent as if each individual publication or patent was specifically and individually incorporated by reference.

From the foregoing description, one skilled in the art can easily ascertain the essential characteristics of this invention; can make various changes and modifications of the invention to adapt it to various usages and conditions. Thus, other embodiments are also within the claims.

What is claimed:

1. A method for identifying an agent that modulates protein aggregation in vitro, said method comprising the steps of:
(a) combining protein molecules or fragments thereof and a candidate agent under conditions allowing for aggregation of said protein molecules; and
(b) determining whether aggregation of said protein molecules or fragments thereof is increased or decreased in comparison to aggregation in the absence of said agent, thereby identifying an agent that modulates protein aggregation in vitro.

2. The method of claim 1, wherein said protein, when present in its conformationally destabilized state in a human, results in a conformational disease.

3. The method of claim 1, wherein said protein is alpha-crystallin.

4. The method of claim 1, wherein said protein is keratin.

5. The method of claim 1, wherein said protein is alpha-synuclein.

6. The method of claim 1, wherein said protein is rhodopsin.

7. The method of claim 1, wherein said protein is a polyglutamine protein.

8. The method of claim 1, wherein said protein is one or more of the following: involucrin, huntingtin, ataxin-1, ataxin-2, ataxin-3, ataxin-7, alpha-1A voltage dependent calcium channel, androgen receptor, cystic fibrosis transmembrane conductance regulator, or atrophin-1.

9. The method of claim 1, wherein said protein is a serpin.

10. The method of claim 1, wherein said protein is antitrypsin.

11. The method of claim 1, wherein said protein is neuroserpin.

12. The method of claim 1, wherein said protein is an amyloid protein.

13. The method of claim 1, wherein said protein is one or more of the following: serum amyloid A, beta-amyloid peptide, lysozyme, fibrinogen a alpha, apolipoprotein A-I, transthyretin, lactadherin, islet amyloid polypeptide, gesolin, atrial natriuretic factor, procalcitonin, cystatin C, beta-2 microglobulin, immunoglobulin light chain, or gamma heavy chain.

14. The method of claim 1, wherein said protein, when present in its conformationally destabilized state in a human, results in a neurological disease.

15. The method of claim 1, wherein said protein is a superoxide dismutase (SOD).

16. The method of claim 15, wherein said SOD includes a mutation.

17. The method of claim 16, wherein said SOD is a mammalian SOD.

18. The method of claim 17, wherein said mammalian SOD is SOD-1.

19. The method of claim 18, wherein said SOD-1 is an apo-SOD-1, a zinc-deficient SOD-1, a holo-SOD-1, or a mutant SOD-1.

20. The method of claim 15, wherein said SOD is human erythrocytic SOD-1.

21. The method of claim 15, wherein said SOD is bovine, equine, porcine or rat SOD.

22. The method of claim 15, wherein said SOD is recombinantly produced.

23. The method of claim 15, wherein said SOD is produced in a bacterial culture, yeast culture, insect cell line culture, or an immortalized human cell culture.

24. The method of claim 1, wherein said aggregation is determined using a light scattering methodology, tryptophan fluorescence, UV absorption, turbidity measurement, a filter retardation assay, size exclusion chromatography, reversed-phase high performance liquid chromatography, an immunological assay, a fluorescent binding assay, a protein-staining assay, microscopy, or polyacrylamide gel electrophoresis (PAGE).

25. The method of claim 1, wherein said protein molecules and said agent are combined in a metal-catalyzing oxidation buffer.

26. The method of claim 25, wherein said metal-catalyzing oxidation buffer is an ascorbate/copper buffer.

27. The method of claim 1, wherein said protein molecules and said agent are combined for at least six hours.

28. The method of claim 1, wherein said protein molecules and said agent are combined at about 37° C.

29. The method of claim 1, wherein said protein molecules and said agent are combined in a well of a microtiter plate.

30. The method of claim 1, wherein said assay is performed using high-throughput robotics.

31. The method of claim 1, wherein aggregation of said protein molecules is decreased.

32. The method of claim 1, wherein aggregation of said protein molecules is increased.

33. The method of claim 1, wherein said agent is further tested in a cell-based or animal model system.

34. A method for identifying an agent that promotes a native conformation of a protein, said method comprising the steps of
a) combining said protein and an agent under a condition that conformationally destabilizes said protein molecule; and
b) determining whether said agent promotes formation of a native conformation of said protein.

35. The method of claim 34, wherein said protein, when present in its conformationally destabilized state in a human, results in a conformational disease.

36. The method of claim 34, wherein said protein is alpha-crystallin.

37. The method of claim 34, wherein said protein is keratin.

38. The method of claim 34, wherein said protein is alpha-synuclein.

39. The method of claim 34, wherein said protein is rhodopsin.

40. The method of claim 34, wherein said protein is a polyglutamine protein.

41. The method of claim 34, wherein said protein is one or more of the following: involucrin, huntingtin, ataxin-1, ataxin-2, ataxin-3, ataxin-7, alpha-1A voltage dependent calcium channel, androgen receptor, cystic fibrosis transmembrane conductance regulator, or atrophin-1.

42. The method of claim 34, wherein said protein is a serpin.

43. The method of claim 34, wherein said protein is antitrypsin.

44. The method of claim 34, wherein said protein is neuroserpin.

45. The method of claim 34, wherein said protein is an amyloid protein.

46. The method of claim 34, wherein said protein is one or more of the following: serum amyloid A, beta-amyloid peptide, lysozyme, fibrinogen a alpha, apolipoprotein A-I, transthyretin, lactadherin, islet amyloid polypeptide, gesolin, atrial natriuretic factor, procalcitonin, cystatin C, beta-2 microglobulin, immunoglobulin light chain, or gamma heavy chain.

47. The method of claim 34, wherein said protein, when present in its conformationally destabilized state in a human, results in a neurological disease.

48. The method of claim 34, wherein said protein is a SOD.

49. The method of claim 48, wherein said SOD is a mammalian SOD.

50. The method of claim 49, wherein said mammalian SOD is SOD-1.

51. The method of claim 50, wherein said SOD-1 is an apo-SOD-1, a zinc-deficient SOD-1, a holo-SOD-1 polypeptide, or a mutant SOD-1.

52. The method of claim 34, wherein said conformationally destabilizing condition involves denaturation.

53. The method of claim 52, wherein said denaturation involves thermally-induced unfolding or aggregation of said protein.

54. The method of claim 52, wherein said denaturation involves chemically-induced unfolding or aggregation of said protein.

55. The method of claim 34, wherein formation of said native conformation of said protein is determined using a light scattering methodology, tryptophan fluorescence, UV absorption, turbidity measurement, a filter retardation assay, size exclusion chromatography, reversed-phase high performance liquid chromatography, an immunological assay, a fluorescent binding assay, a protein-staining assay, microscopy, or polyacrylamide gel electrophoresis (PAGE).

56. The method of claim 34, wherein formation of said native conformation of said protein is determined by assaying for soluble protein.

57. The method of claim 34, wherein said agent is further tested in a cell-based or animal model system.

58. A method for identifying an agent that promotes a native conformation of a SOD protein, said method comprising the steps of
a) contacting said SOD protein and an agent under conditions wherein said SOD protein is in its native conformation; and
b) determining whether said agent binds to said SOD in its native state, thereby identifying an agent that promotes the native conformation of the SOD protein.

59. The method of claim 58, wherein said SOD is a mammalian SOD.

60. The method of claim 59, wherein said mammalian SOD is SOD-1.

61. The method of claim 58, wherein said binding is assayed using a Biacore measurement.

62. The method of claim 58, wherein said binding is measured using a radio-, fluorescently-, or biotin-labeled agent.

63. The method of claim 58, further comprises testing said agent in a cell-based or animal model system.

64. A method for identifying an agent that modulates protein aggregation of a protein in a cell, said method comprising the steps of
a) providing a cell line which produces said protein and an agent under conditions allowing for aggregation of said protein in said cell line; and
b) determining whether aggregation of said protein in said cell line is increased or decreased in comparison to aggregation in the absence of said agent, thereby identifying an agent that modulates protein aggregation in said cell line.

65. The method of claim 64, wherein said protein, when present in its conformationally destabilized state in a human, results in a conformational disease.

66. The method of claim 64, wherein said protein is alpha-crystallin.

67. The method of claim 64, wherein said protein is keratin.

68. The method of claim 64, wherein said protein is alpha-synuclein.

69. The method of claim 64, wherein said protein is rhodopsin.

70. The method of claim 64, wherein said protein is a polyglutamine protein.

71. The method of claim 64, wherein said protein is one or more of the following: involucrin, huntingtin, ataxin-1, ataxin-2, ataxin-3, ataxin-7, alpha-1A voltage dependent calcium channel, androgen receptor, cystic fibrosis transmembrane conductance regulator, or atrophin-1.

72. The method of claim 64, wherein said protein is a serpin.

73. The method of claim 64, wherein said protein is antitrypsin.

74. The method of claim 64, wherein said protein is neuroserpin.

75. The method of claim 64, wherein said protein is an amyloid protein.

76. The method of claim 64, wherein said protein is one or more of the following: serum amyloid A, beta-amyloid peptide, lysozyme, fibrinogen a alpha, apolipoprotein A-I, transthyretin, lactadherin, islet amyloid polypeptide, gesolin, atrial natriuretic factor, procalcitonin, cystatin C, beta-2 microglobulin, immunoglobulin light chain, or gamma heavy chain.

77. The method of claim 64, wherein said protein, when present in its conformationally destabilized state in a human, results in a neurological disease.

78. The method of claim 64, wherein said protein is a SOD.

79. The method of claim 78, wherein said SOD is a mammalian SOD.

80. The method of claim 79, wherein said mammalian SOD is SOD-1.

81. The method of claim 80, wherein said SOD-1 is overexpressed in said cell line.

82. The method of claim 80, wherein said SOD-1 is a mutant SOD-1.

83. The method of claim 64, wherein said cell line is treated with a substance that decreases degradation of the protein.

84. The method of claim 83, wherein said substance is a proteasome inhibitor.

85. The method of claim 64, wherein said cell line is a HEK293, COS, 3T3, or HeLa cell line.

86. The method of claim 64, wherein said aggregation is determined using immunological detection or a biochemical assay.

87. The method of claim 64, wherein said agent is further tested in a cell-based or animal model system.

88. A method of identifying an agent for treating a disorder resulting from the presence of a conformationally destabilized protein, said method comprising the step of:
a) administering a therapeutically effective amount of an agent identified in any one of claims 1, 33, 34, 57, 58, 63, 64, 87, 88, or 109 to an animal in which a conformationally destabilized protein is present that results in a disease;
b) determining whether the agent decreases a disease symptom associated with expression of the conformationally destabilized protein, a decrease in the symptom as compared to control animals indicating that the agent is a useful pharmaceutical for treating the disease.

89. The method of claim 88, wherein said protein, when present in its conformationally destabilized state in a human, results in a conformational disease.

90. The method of claim 88, wherein said protein is alpha-crystallin.

91. The method of claim 88, wherein said protein is keratin.

92. The method of claim 88, wherein said protein is alpha-synuclein.

93. The method of claim 88, wherein said protein is rhodopsin.

94. The method of claim 88, wherein said protein is a polyglutamine protein.

95. The method of claim 88, wherein said protein is one or more of the following: involucrin, huntingtin, ataxin-1, ataxin-2, ataxin-3, ataxin-7, alpha-1A voltage dependent calcium channel, androgen receptor, cystic fibrosis transmembrane conductance regulator, or atrophin-1.

96. The method of claim 88, wherein said protein is a serpin.

97. The method of claim 88, wherein said protein is antitrypsin.

98. The method of claim 88, wherein said protein is neuroserpin.

99. The method of claim 88, wherein said protein is an amyloid protein.

100. The method of claim 88, wherein said protein is one or more of the following: serum amyloid A, beta-amyloid peptide, lysozyme, fibrinogen a alpha, apolipoprotein A-I, transthyretin, lactadherin, islet amyloid polypeptide, gesolin, atrial natriuretic factor, procalcitonin, cystatin C, beta-2 microglobulin, immunoglobulin light chain, or gamma heavy chain.

101. The method of claim 88, wherein said protein, when present in its conformationally destabilized state in a human, results in a neurological disease.

102. The method of claim 88, wherein said disease is human ALS or the corresponding ALS-like disease in an animal.

103. The method of claim 88, wherein the animal is a rodent

104. The method of claim 88, wherein said animal is a transgenic rodent.

105. The method of claims 103 or 104, wherein said rodent or transgenic rodent overexpresses said protein.

106. The method of claim 88, wherein said protein is a mutant form of a protein.

107. The method of claim 88, wherein said protein is SOD.

108. The method of claim 107, wherein said SOD is SOD-1.

109. The method of claim 88, wherein said agent is further tested in a cell-based or animal model system.

110. A method of treating a human subject for a disease state associated with possession of a conformationally destabilized protein, comprising administering to said human subject a therapeutically effective amount of one or more of the agents identified in any of the aforementioned screening assays 1, 33, 34, 57, 58, 63, 64, 87, 88, or 109.

111. The method of claim 110, wherein said disease state is a conformational disease.

112. The method of claim 110, wherein said disease state is one or more of the following: Huntington's disease, Parkinson's disease, Alzheimer's disease, cystic fibrosis, Pick's disease, Spinocerebellar ataxia 1, Spinocerebellar ataxia 2, Spinocerebellar ataxia 3, Spinocerebellar ataxia 6, Spinocerebellar ataxia 7, Spinobulbar muscular atrophy, denatorubro-pallidoluysian atrophy, cataracts, cirrohosis, emphysema, hereditary cardiac amyloidosis, Finnish type familial amyloidosis, familial amyloid polyneuropathy, familial amyloid cardiomyopathy, senile systemic amyloidosis, senescence, familial dementia, Type II diabetes, immunoglobulin amyloidosis, white sponge naevus, chronic inflammatory disease, Systemic Amyloidosis (ALys), Systemic Amyloidosis (AFib), Systemic Amyloidosis (AA) (secondary), dialysis-associated amyloidosis, senile cardiac atria amyloidosis, medullary carcinoma thyroid endocrine amyloidosis, Systemic Vascular Amyloidosis HCHWA (Iceland), or Retinitis pigmentosa.

113. The method of claim 110, wherein said disease state is a neurological disease.

114. The method of claim 113, wherein said neurological disease is ALS.

115. An agent identified according to any one of the methods of claims 1, 33, 34, 57, 58, 63, 64, 87, 88, or 109.
